(19) United States
(12) Patent Application Publication
(10) Pub. No.: US 2017/0320908 A1
(43) Pub. Date: Nov. 9, 2017

(54) SOLID PHASE SYNTHESIS OF CYCLIC AMINO ACID MOLECULES

(71) Applicants: ENCYCLE THERAPEUTICS, Toronto (CA); GOVERNING COUNCIL OF THE UNIVERSITY OF TORONTO, Toronto (CA); UNIVERSITE DE SHERBROOKE, Sherbrooke (CA)

(72) Inventors: Jennifer L. Hickey, Toronto (CA); John Mancuso, Rosemere (CA); Eric Marsault, Sherbrooke (CA); Andrew L. Roughton, Port Hope (CA); Adam P. Treder, Przyjazn (PL); Marie-Claude J. Trembley, Sherbrooke (CA); Andrei K. Yudin, Oakville (CA); Serge Zaretsky, Georgetown (CA)

(21) Appl. No.: 15/528,057

(22) PCT Filed: Nov. 17, 2015
(86) PCT No.: PCT/IB2015/058903

§ 371 Date: May 18, 2017

Related U.S. Application Data
(60) Provisional application No. 62/081,780, filed on Nov. 19, 2014.

(51) Int. Cl. C07K 1/107; C07K 5/12; C07K 7/64

(52) U.S. Cl. CPC C07K 1/1077; C07K 7/64; C07K 5/123; C07K 5/126

(57) ABSTRACT

The present invention relates to cyclic amino acid molecules and methods of preparing the same, and in particular the macrocyclization of amino acids or linear peptides bound to a solid support.

### FIELD

The present invention relates to cyclic amino acid molecules and methods of preparing the same, and in particular the macrocyclization of amino acids or linear peptides bound to a solid support.

### BACKGROUND

Macrocycles are large cycles composed of 12 or more atoms. In terms of physicochemical and biological properties, macrocycles are considered an intermediate class between small molecules and large biomolecules. Indeed, with molecular weights usually ranging between 500 and 2,000 Da, they combine large surface areas, a property usually associated with biomolecules such as antibodies, with the potential to reach suitable physicochemical properties conducive to oral bioavailability, which is usually associated with small molecules. Owing to these properties, macrocycles have attracted an increasing level of attention recently as a class belonging to an underexploited chemical space (for recent reviews, see [1-7]). Macrocycles have been used successfully as drug candidates on most target classes, and owing to the aforementioned properties, expectations are high for the modulation of protein-protein interactions, which are characterized by large interacting surface areas and have proven difficult to tackle with small molecules.[8, 9] Chemically, the vast majority of the ˜70 macrocyclic drugs or clinical candidates belong to two families, natural products and peptides.[5]

Several approaches have been reported for the synthesis of chemically and conformationally diverse macrocycles and their subsequent use in medicinal chemistry. Some of these have become the platforms of emerging companies (for specific examples, see [10-12]; for reviews, see [3, 4]). One challenge inherent to the macrocyclization reaction is the ability to bring the two extremities of the linear precursor in close proximity to enable ring formation. Fundamentally, macrocyclization is a unimolecular, largely entropy-driven reaction in which backbone flexibility and transannular interactions represent critical factors that can favour or disfavour the reaction.[13-15] As a result, macrocyclizations are often performed under high or pseudo-high dilution conditions to reduce the formation of undesired dimers and other oligomers.

The Yudin group recently reported a novel class of amphoteric reagents in the form of aziridine aldehyde dimers, and demonstrated their capability to disrupt the three-component Ugi reaction. These reagents are also described by the Yudin group in WO 2008/046232.

When aziridine aldehyde dimers were reacted in the presence of an isocyanide and a linear peptide possessing a free amino and carboxylate functions, aziridine amide-containing peptide macrocycles were isolated.[4, 16-18] One of the distinguishing features of this particular macrocyclization is the unusually high concentration at which it remains productive (typically >0.1 M), which constitutes a distinct preparative advantage compared to other macrocyclization reactions, which are usually performed at mM concentrations. The rationale behind this efficiency has been preliminarily attributed to the maintenance, throughout each intermediate step, of a stabilizing ion pair that facilitates chain folding and keeps the two extremities in close proximity [19] The resulting endocyclic aziridine amide can be opened post-macrocyclization with a variety of nucleophiles, which adds an extra point of diversity to introduce exocyclic substituents. [20-22] Macrocyclization of peptides using aziridine aldehydes is also described by the Yudin group in WO 2010/105363.

### SUMMARY OF THE INVENTION

In an aspect, there is provided a process to produce a cyclic molecule bound to a solid support comprising reacting a peptide, having an amino terminus and a carboxyl terminus and bound to the solid support by a side chain of the peptide, with an isocyanide and a compound having formula (Ia) and/or (Ib):

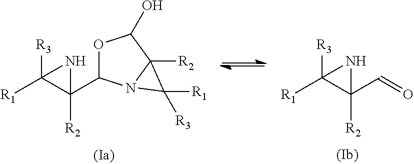

wherein:
R₁, R₂ and R₃ are independently selected from H; lower alkyl; alkenyl; heterocycle; cyckoalkyl; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -lower alkyl-aryl, or —NRₐR_{b}, where Rₐ and R_{b} are independently selected from H, lower alkyl, aryl or -lower alkyl-aryl; —C(O)R_{c}, wherein R_{c} is selected from lower alkyl, aryl or -lower alky-aryl; or -lower alkyl-OR_{d}, wherein R_{d} is a suitable protecting group or OH group; all of which are optionally substituted at one or more substitutable positions with one or more suitable substituents; and
the aldehyde component thereof may optionally be in its bisulfite adduct form;
and the compound comprises an aziridine chiral center proximal to the aldehyde with stereochemistry that is homochiral with respect to the carbon atom proximal to the amino terminus of the peptide.
   In an aspect, there is provided a process for preparing a cyclic molecule of formula [(III)] bound to a solid support:

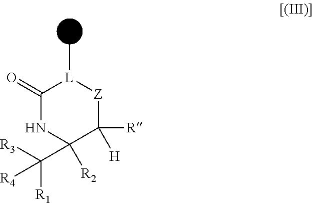

wherein,
R₁, R₂ and R₃ are independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b}, are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents,
Z is an amino terminus of a peptide;
C═O— is the carboxy terminus of the peptide;
L, along with Z and —C═O— is the peptide;
R″ is an optionally substituted amide;
R₄ is a nucleophile;
wherein the peptide is bound to the solid support by a side chain of the peptide;
   comprising reacting a compound having formula [(II)] bound to the solid support with a nucleophile:

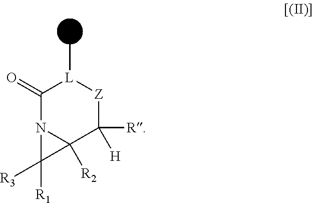

In an aspect, there is provided a process for preparing a cyclic molecule of formula [(IV)]:

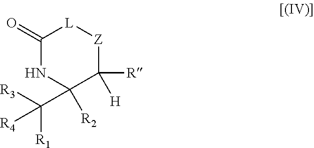

wherein,
R₁, R₂ and R₃ are independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents;
Z is an amino terminus of a peptide;
—C═O— is the carboxy terminus of the peptide;
L, along with Z and —C═O— is the peptide;
R″ is an optionally substituted amide;
R₄ is a nucleophile;
   comprising cleaving a solid support from a cyclic molecule of formula [(III)] bound to the solid support:

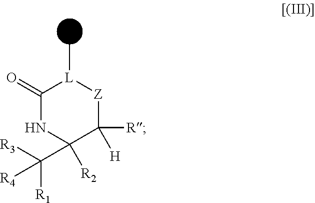

and optionally deprotecting one or more side chains of the peptide if one or more of said side chains are protected with protecting groups.

In an aspect, there is provided a cyclic molecule of formula [(IV)]:

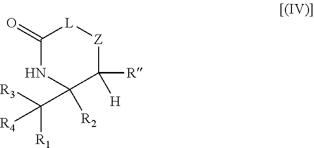

wherein,
R₂ is independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents;
Z is an amino terminus of a peptide;
—C═O— is the carboxy terminus of the peptide;
L, along with Z and —C═O— is the peptide;
R₁, R₃ and R₄ are H;
R″ is an optionally substituted amide;

### BRIEF DESCRIPTION OF THE FIGURES

Embodiments of the invention may best be understood by referring to the following description and accompanying drawings. In the description and drawings, like numerals refer to like structures or processes. In the drawings:

FIG. 1 is a general synthetic approach (exemplified with all-L-amino acids and (S)-aziridine aldehyde dimer derived from L-Scr).

FIG. 2 shows ¹H-¹H TOCSY NMR of compound 25. The linker region is annotated and highlighted in red on the structure. The corresponding cross-peaks from the new NH to the adjacent linker atoms are highlighted on the TOCSY spectrum

FIG. 3 shows Scheme 1: strategies for side chain attachment of the C-terminal amino acid.

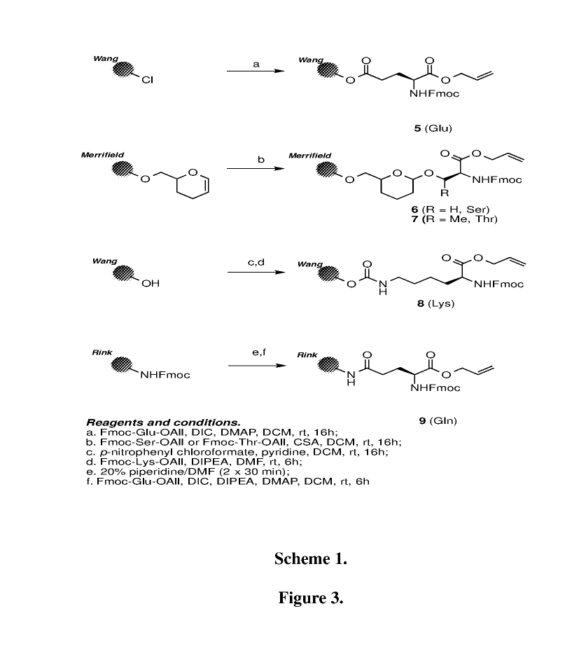

FIG. 4 shows Scheme 2: detailed synthetic scheme, exemplified for the synthesis of macrocycle 14.

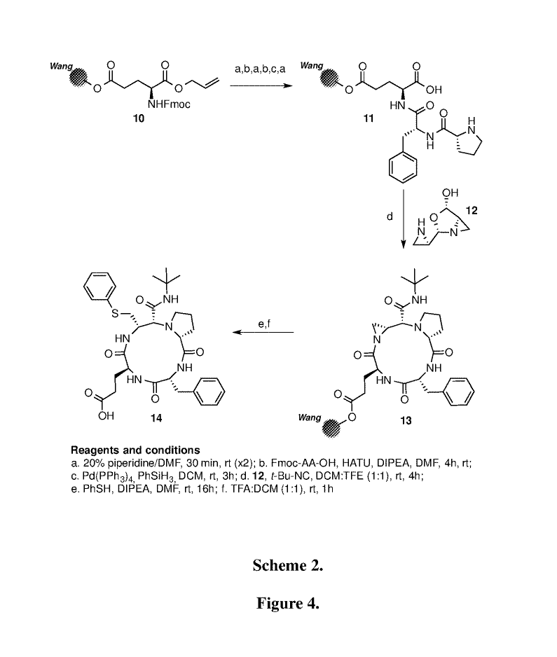

Table 1 shows macrocycles synthesized on solid phase by three-component coupling.

Spectra and Chromatograms of macrocycles are also provided

### DETAILED DESCRIPTION

In order to further exploit the potential of macrocyclization, there is reported herein a solid-phase approach. Solid-phase synthesis has amply demonstrated its potential in both high throughput parallel synthesis or split-pool combinatorial chemistry.[23, 24] It constitutes a tool of choice to build libraries suitable for hit identification as well as rapid analog generation for hit-to-lead optimization. Building on our recent application of the disrupted Ugi reaction with aziridine aldehyde dimers to the solid-phase synthesis of piperazinones, we report herein the development of a solid-phase macrocyclization protocol and aziridine ring-opening strategy.[25]

In an aspect, there is provided a process to produce a cyclic molecule bound to a solid support comprising reacting a peptide, having an amino terminus and a carboxyl terminus and bound to the solid support by a side chain of the peptide, with an isocyanide and a compound having formula (Ia) and/or (Ib):

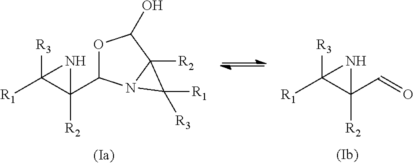

wherein:
R₁, R₂ and R₃ are independently selected from H; lower alkyl; alkenyl; heterocycle; cyckoalkyl; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -lower alkyl-aryl, or —NRₐR_{b}, where Rₐ and R_{b} are independently selected from H, lower alkyl, aryl or -lower alkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents; and
aldehyde component thereof may optionally be in its bisulfate adduct form;
and the compound comprises an aziridine chiral center proximal to the aldehyde with stereochemistry that is homochiral with respect to the carbon atom proximal to the amino terminus of the peptide.

As used herein, the term “amino acid molecule” is meant to include single amino acids and also peptides.

As used herein, the term “amino acid” refers to molecules containing an amine group, a carboxylic acid group and a side chain that varies. Amino acid is meant to include not only the twenty amino acids commonly found in proteins but also non-standard amino acids and unnatural amino acid derivatives known to those of skill in the art, and therefore includes, but is not limited to, alpha, beta and gamma amino acids. Peptides are polymers of at least two amino acids and may include standard, non-standard, and unnatural amino acids.

Although in certain embodiments, cyclization of peptides are described, a person skilled in the art would understand based on the present description that the described methods could also be applied to cyclize a single amino acid. For example, such cyclization would result in piperazinones.

The term “suitable substituent” as used in the context of the present invention is meant to include independently H; hydroxyl; cyano; alkyl, such as lower alkyl, such as methyl, ethyl, propyl, n-butyl, t-butyl, hexyl and the like; alkoxy, such as lower alkoxy such as methoxy, ethoxy, and the like; aryloxy, such as phenoxy and the like; vinyl; alkenyl, such as hexenyl and the like; alkynyl; formyl; haloalkyl, such as lower haloalkyl which includes CF₃, CCl₃ and the like; halide; aryl, such as phenyl and napthyl; heteroaryl, such as thienyl and furanyl and the like; amide such as C(O)NRₐR_{b}, where Rₐ and R_{b} are independently selected from lower alkyl, aryl or benzyl, and the like; acyl, such as C(O—C₆H₅, and the like; ester such as —C(O)OCH₃ the like; ethers and thioethers, such as O-Bn and the like; thioalkoxy; phosphino; and —NRₐR_{b}, where Rₐ and R_{b} are independently selected from lower alkyl, aryl or benzyl, and the like. It is to be understood that a suitable substituent as used in the context of the present invention is meant to denote a substituent that does not interfere with the formation of the desired product by the processes of the present invention.

As used in the context of the present invention, the term “lower alkyl” as used herein either alone or in combination with another substituent means acyclic, straight or branched chain alkyl substituent containing from one to six carbons and includes for example, methyl, ethyl, 1-methylethyl, 1-methylpropyl, 2-methylpropyl, and the like. A similar use of the term is to be understood for “lower alkoxy”, “lower thioalkyl”, “lower alkenyl” and the like in respect of the number of carbon atoms. For example, “lower alkoxy” as used herein includes methoxy, ethoxy, t-butoxy.

The term “alkyl” encompasses lower alkyl, and also includes alkyl groups having more than six carbon atoms, such as, for example, acyclic, straight or branched chain alkyl substituents having seven to ten carbon atoms.

The term “aryl” as used herein, either alone or in combination with another substituent, means an aromatic monocyclic system or an aromatic polycyclic system. For example, the term “aryl” includes a phenyl or a napthyl ring, and may also include larger aromatic polycyclic systems, such as fluorescent (e.g. anthracene) or radioactive labels and their derivatives.

The term “heteroaryl” as used herein, either alone or in combination with another substituent means a 5, 6, or 7-membered unsaturated heterocycle containing from one to 4 heteroatoms selected from nitrogen, oxygen, and sulphur and which form an aromatic system. The term “heteroaryl” also includes a polycyclic aromatic system comprising a 5, 6, or 7-membered unsaturated heterocycle containing from one to 4 heteroatoms selected from nitrogen, oxygen, and sulphur.

The term “cycloalkyl” as used herein, either alone or in combination with another substituent, means a cycloalkyl substituent that includes for example, but is not limited to, cyclopropyl, cyclobutyl, cyclopentyl, cyclohexyl and cycloheptyl.

The term “cycloalkyl-alkyl-” as used herein means an alkyl radical to which a cycloalkyl radical is directly linked; and includes, but is not limited to, cyclopropylmethyl, cyclobutylmethyl, cyclopentylmethyl, 1-cyclopentylethyl, 2-cyclopentylethyl, cyclohexylmethyl, 1-cyclohexylethyl and 2-cyclohexylethyl. A similar use of the “alkyl” or “lower alkyl” terms is to be understood for aryl-alkyl-, aryl-loweralkyl- (e.g. benzyl), -lower alkyl-alkenyl (e.g. allyl), heteroaryl-alkyl-, and the like as used herein. For example, the term “aryl-alkyl-” means an alkyl radical, to which an aryl is bonded. Examples of aryl-alkyl- include, but are not limited to, benzyl (phenylmethyl), 1-phenylethyl, 2-phenylethyl and phenylpropyl.

As used herein, the term “heterocycle”, either alone or in combination with another radical, means a monovalent radical derived by removal of a hydrogen from a three- to seven-membered saturated or unsaturated (including aromatic) heterocycle containing from one to four heteroatoms selected from nitrogen, oxygen and sulfur. Examples of such heterocycles include, but are not limited to, aziridine, epoxide, azetidine, pyrrolidine, tetrahydrofuran, thiazolidine, pyrrole, thiophene, hydantoin, diazepine, imidazole, isoxazole, thiazole, tetrazole, piperidine, piperazine, homopiperidine, homo-piperazine, 1,4-dioxane, 4-morpholine, 4-thiomorpholine, pyridine, pyridine-N-oxide or pyrimidine, and the like.

The term “alkenyl”, as used herein, either alone or in combination with another radical, is intended to mean an unsaturated, acyclic straight chain radical containing two or more carbon atoms, at least two of which are bonded to each other by a double bond. Examples of such radicals include, but are not limited to, ethenyl (vinyl), 1-propenyl, 2-propenyl, and 1-butenyl.

The term “alkynyl”, as used herein is intended to mean an unsaturated, acyclic straight chain radical containing two or more carbon atoms, at least two of which are bonded to each other by a triple bond. Examples of such radicals include, but are not limited to, ethynyl, 1-propynyl, 2-propynyl, and 1-butynyl.

The term “alkoxy” as used herein, either alone or in combination with another radical, means the radical —O—(C₁₋ₙ)alkyl wherein alkyl is as defined above containing 1 or more carbon atoms, and includes for example methoxy, ethoxy, propoxy, 1-methylethoxy, butoxy and 1,1-dimethylethoxy. Where n is 1 to 6, the term “lower alkoxy” applies, as noted above, whereas the term “alkoxy” encompasses “lower alkoxy” as well as alkoxy groups where n is greater than 6 (for example, n =7 to 10). The term “aryloxy” as used herein alone or in combination with another radical means —O-aryl, wherein aryl is defined as noted above.

In some embodiments, the amino terminus of the peptide is a secondary amino group.

In some embodiments, any one of R₁-R₃ is H, preferably each of R₁-R₃ is H.

In some embodiments, R₂ and R₃ are H.

In some embodiments, R₁ is CH₂OTBDMS or CH_{2 Hu i}Pr.

In some embodiments, the isocyanide is selected from the group consisting of: (S)-(−)-α-Methylbenzyl isocyanide; 1,1,3,3,-Tetramethylbutyl isocyanide; 1-Pentyl isocyanide; 2,6-Dimethylphenyl isocyanide; 2-Morpholinoethyl isocyanide; 2-Naphthyl isocyanide; 2-Pentyl isocyanide; 4-Methoxyphenyl isocyanide; Benzyl isocyanide; Cutyl isocyanide; Cyclohexyl isocyanide; Isopropyl isocyanide; p-Toluenesulfonylmethyl isocyanide; Phenyl isocyanide dichloride; tert-Butyl isocyanide; (Trimethylsilyl)methyl isocyanide; 1H-Benzotriazol-1-ylmethyl isocyanide; 2-Chloro-6-methylphenyl isocyanide; Di-tert-butyl 2-isocyanosuccinate; tert-Butyl 2-isocyano-3-methylbutyrate; tert-Butyl 2-isocyano-3-phenylpropionate; tert-Butyl 2-isocyanopropionate; and tert-Butyl 3-isocyanopropionate; tert-Butyl isocyanoacetate; and ethyl isocyanoacetate.

In some embodiments, the isocyanide is tert-Butyl isocyanide.

In some embodiments, the peptide is between 2 and 30 amino acids in length, preferably between 2 and 9 amino acids in length.

In some embodiments, the cyclic molecule bound to the solid support is of formula [(II)]:

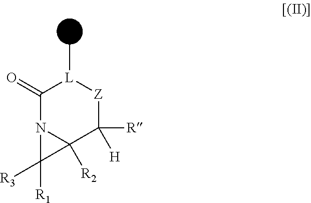

wherein,

Z is an amino terminus of the peptide;

—C═O— is the carboxy terminus of the peptide; and

L, along with Z and —C═O— is the peptide;

R″ is an optionally substituted amide.

In an aspect, there is provided a process for preparing a cyclic molecule of formula [(III)] bound to a solid support:

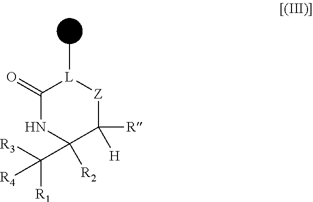

wherein,
R₁, R₂ and R₃ are independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents,
Z is an amino terminus of a peptide;
—C═O— is the carboxy terminus of the peptide;
L, along with Z and —C═O— is the peptide;
R″ is an optionally substituted amide;
R₄ is a nucleophile;
wherein the peptide is bound to the solid support by a side chain of the peptide;
   comprising reacting a compound having formula [(II)] bound to the solid support with a nucleophile:

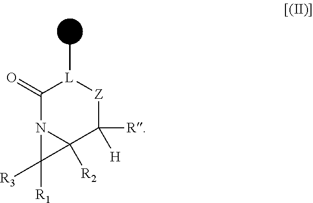

In an aspect, there is provided a process for preparing a cyclic molecule of formula [(IV)]:

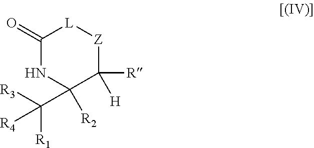

wherein,
R₁, R₂ and R₃ are independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents;
Z is an amino terminus of a peptide;
—C═O— is the carboxy terminus of the peptide;
L, along with Z and —C═O— is the peptide;
R″ is an optionally substituted amide;
R₄ is a nucleophile;
   comprising cleaving a solid support from a cyclic molecule of formula [(III)] bound to the solid support:

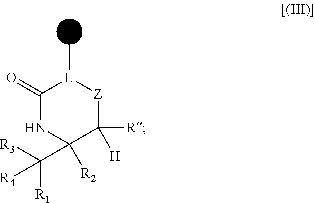

and optionally deprotecting one or more side chains of the peptide if one or more of said side chains are protected with protecting groups.

Suitable protecting groups would be well understood by a person skilled in the art. Without limitation, preferable protecting groups include Fmoc, Boc, Alloc, Ddz, and Bpoc.

In some embodiments, the solid support is a resin. In some embodiments, the resin is selected from the group consisting of Wang, MBHA , HMPA, Tentagel, Trityl, 2′-Chlorotrityl, Argogel, PS-PEG, ChemMatrix, PEG support, Mimotopes' Lanterns.

In some embodiments, the peptide is elongated prior to cyclization. In some embodiments, elongation is by Fmoc chemistry.

In some embodiments, the process further comprises ring-opening of the aziridine moiety with a nucleophile.

In some embodiments, the process further comprises conjugating a fluorescent tag to the cyclic molecule by nucleophilic ring-opening of the aziridine moiety.

In some embodiments, the nucleophile is selected from the group consisting of: R—C(O)SH, Ar—C(O)SH, Ar—SH, H₂, R—SH, RS—, N₃—, R₃P, NC—, I—, Ar—NH2, Br—, R—CO2H; preferentially, the nucleophile is selected from the group consisting of: R—C(O)SH, Ar—C(O)SH, Ar—SH, H₂, N₃

In some embodiments, the nucleophilic ring-opening of the aziridine moiety is carried out using a soft, highly polarizable, low-electronegativity nucleophile having pKa<15.

In some embodiments, the process further comprises cleavage of the solid support from the cyclic molecule.

In some embodiments, the process further comprises deprotecting one or more side chains of the amino acid molecule if one or more of said side chains are protected with protecting groups.

In some embodiments, the peptide is a linear peptide.

In some embodiments, the amino terminus amino acid of the linear peptide is selected from the group consisting of proline and an amino acid with an amino group substituted with H and Bn, with H and CH2CH2SO2Ph, with H and CH2CH2CN, or with H and CH3.

In some embodiments, the amino acids of the peptide are D or L amino acids selected from the group consisting of: alanine, arginine, asparagine, aspartic acid, cysteine, glutamic acid, glutamine, glycine, histidine, isoleucine, leucine, lysine, methionine, phenylalanine, proline, selenocysteine, serine, tyrosine, threonine, tryptophan and valine.

In some embodiments, the amino acids of the peptide are alpha-amino acids.

In some embodiments, the amino acids of the peptide are beta-amino acids.

In some embodiments, the amino acids of the peptide are gamma-amino acids.

In some embodiments, the solid support is bound to the side chain of the carboxy terminus of the peptide.

In some embodiments, the side chains of the peptide are polar side chains.

In some embodiments, the side chains of the peptide are non-polar side chains.

In some embodiments, the side chain of the amino acid molecule is selected from the group consisting of: glutamic acid, glutamine, serine, threonine, lysine, aspartic acid, asparagine, homo-serine, ornithine, 2,4-diaminobutyric acid, 2,3-diaminopropionic acid, tyrosine, tryptophan, histidine, and 4-hydroxy-proline.

In an aspect, there is provided a cyclic molecule of formula [(IV)]:

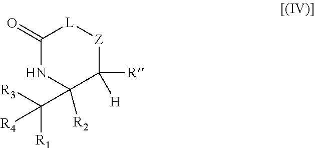

wherein,
R₂ is independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents;
Z is an amino terminus of a peptide;
—C═O— is the carboxy terminus of the peptide;
L, along with Z and —C═O— is the peptide;
R₁, R₃ and R₄ are H;
R″ is an optionally substituted amide;

In some embodiments, the cyclic molecule is not:

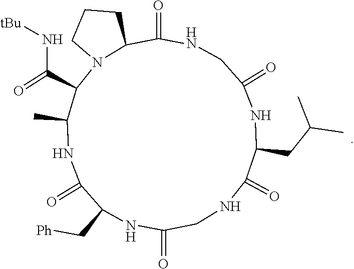 [SMILES: CC(C)C[C@@H]1NC(=O)CNC(=O)[C@@H]2CCCN2[C@H](C(=O)NC(C)(C)C)[C@H](C)NC(=O)[C@H](Cc2ccccc2)NC(=O)CNC1=O]

The following examples are illustrative of various aspects of the invention, and do not limit the broad aspects of the invention as disclosed herein.

### EXAMPLES

### Materials and Methods

Preparation of Fmoc-Glu-OAll anchored to Wang resin via side chain—Wang resin 0.95 mmol/g (9 g, 8.55 mmol, 1 eq.) was swollen in 150 mL DCM in a 250 mL glass solid phase peptide synthesis reactor equipped with a fritted glass funnel. The resin was then further washed with DCM (2×150 mL, 4 min. cycle on an orbital shaker). Fmoc-L-Glu-OAll (8.75 g, 21.375 mmol, 2.5 eq.) and DMAP (157 mg, 1.28 mmol, 0.15 eq.) in 150 mL of DCM were added to the resin, followed by DIC (3.31 mL, 21.375 mmol, 2.5 eq.). The reaction mixture was shaken overnight on an orbital shaker then drained and washed with DCM, MeOH, DCM, MeOH, DCM (3×150 mL each, 4 min cycles). The resin was capped with acetic anhydride (3.2 mL, 34.2 mmol, 3 eq.) in the presence of pyridine (2.75 mL, 34.2 mmol, 3 eq.) in 150 mL of dry DCM. Finally the resin was drained, washed (DCM, MeOH, DCM, MeOH, DCM—3×150 mL each, 4 min cycles) and dried in vacuo. Loading was confirmed by mini-cleavage (TFA:DCM 1:1, 1.5 h) and was close to the manufacturer's specifications (0.68 mmol of Fmoc-L-Glu-OAll/g).

Fmoc deprotection—0.3 mmol of resin (0.441 mg) was transferred into a 10 mL polypropylene disposable reactors. 8 mL of 20% piperidine/DMF was added and the resin was shaken for 30 minutes. The resin was then drained and the above operation repeated once. Finally the resin was drained and washed with DMF (5×8 mL).

Anchoring of Fmoc-D-Phe followed by addition of Fmoc-D-Pro—Fmoc-D-Phe-OH (349 mg, 0.9 mmol, 3 eq.) and HATU (291 mg, 0.765 mmol, 2.55 eq.) were dissolved in DMF (4 mL). DIPEA (266 μL, 1.53 mmol, 5.1 eq.) was added and the mixture was transferred to the resin. After four hours of agitation, the resin was drained and washed with DMF (2×), IPA, DMF, DCM (3×), diethyl ether (8 mL, 4 min cycles). The Fmoc group was deprotected using 20% piperidine as described above, then Fmoc-D-Pro-OH was introduced similarly to Fmoc-D-Phe-OH.

Allyl ester deprotection—Resin (0.1 mmol, 1 eq.) was washed with dry DCM (8 mL). Phenylsilane (123 uL, 1 mmol, 10 eq.) in 2 mL of dry DCM was added, followed by Pd(Ph₃P)₄ (35 mg, 0.03 mmol, 0.3 eq.) in 2 mL of dry DCM. The resin was agitated for three hours then the dark-colored solution was drained. The resin was washed with 0.5% sodium diethyl dithiocarbamate/DMF (3×), isopropanol, DMF, DCM (3×), diethyl ether (8 mL, 3-4 min cycles), then dried in vacuo.

Fmoc deprotection, macrocyclization and nucleophilic ring-opening of aziridine—the above resin (0.1 mmol) was swollen in DMF (30 min), drained and then 8 mL of 20% piperidine/DMF was added. After 30 minutes of agitation, the resin was drained and again 20% piperidine/DMF was added. The resin was agitated for 30 min, drained and washed with DMF (5x) then DCM (5×; 8 mL, 4 min cycles) and finally with dry DCM:TFE 1:1 (3×; 3 mL, 4 min cycles). Next 1 mL of dry DCM was added, followed by a 0.1 M solution of (S)-aziridine aldehyde dimer in TFE (1 mL, 0.1 mmol, 1 eq.). The resin was agitated for one minute, then t-butyl isocyanide (22.6 μL, 0.2 mmol, 2 eq.), was added. The reaction mixture was agitated for four more hours then drained and washed with DCM (4×8 mL; 4 min cycles). The resin was further washed with DMF (4×8 mL; 4 min cycles), then 2.5 mL DMF was added, followed by PhSH (113 μL, 1.1 mmol, 11 eq.) and DIPEA (174 μL, 1.0 mmol, 10 eq.). The reaction mixture was agitated overnight then drained and washed with DMF, DCM, MeOH, DCM, MeOH, DCM (3× each; 8 mL, 4 min cycles).

Resin cleavage—The final macrocycle was cleaved with 50% TFA/DCM (6 mL) for 75 minutes. The resin was then washed 4× with 50% TFA/DCM. Combined filtrates were evaporated and purified by preparative HPLC (14-30) or MS-triggered preparative HPLC (31-35) MS-triggered preparative HPLC. Fractions containing the product were re-analyzed by UPLC-MS, pooled and the product was isolated by lyophilization.

Materials—Resins were purchased from RAPP Polymere and used as received. Amino acids, TFA and DIPEA were purchased from CHEM-IMPEX and used as received. DCM was distilled over calcium hydride, THF over Na/benzophenone, DMF was stored over pre-activated 3 Å molecular sieves. All other reagents were purchased from commercial sources and used without additional purification. Reactions were performed in 10 mL polypropylene cartridge syringe reactors with caps (Applied Separations), One-Way Teflon Stopcocks (Applied Seperations). 10 mL reactors were shaken vertically (˜800 rpm) on an IKA VIBRAX-VXR orbital shaker. Alternatively, Mettler-Toledo 24-well blocks (Silicycle Inc.) were used.

A person skilled in the art would understand that alternate reagents could be used without departing from the methods described herein. For example, without limitation: suitable acids could include TFA, HCl, HBr, HF, and methanesulfonic acid; suitable co-solvents could include DCM, dioxane, DCE, and EtOAc; suitable additives could include thiophenol, thioanisole, water, Et₃SiH, iPr₃SiH, thiocresol, cresol, dithiothreitol, ethanedithiol, phenol, alkanethiols, anisole, indole, and various thioethers; suitable coupling reagents could include HBTU, HATU, TBTU, HCTU series, the BOP series (PyBOP, PyBrOP, PyAOP . . . etc.), DEPBT, T3P, DIC or EDCI+ additives (HOBt, HOAt, 30ClHOBt), DPPA, and Mukayama's reagent; as further discussed in El-Faham & Albericio, Chem. Rev. 2011, 6557.

Compound Characterization—Cleavage products and final products 14-30 were analysed on a Waters Alliance 2695 HPLC with UV detection at 214 and 254 nm equipped with an ACE 5 C18 100×2.1 mm column. MS spectra were recorded on a Waters Micromass ZQ2000 (electrospray) instrument. The following gradient conditions were used on this apparatus.

Analytical method: (A: 0.1% HCOOH in H₂O, B: 0.1% HCOOH in ACN):

Cleavage products and final products 31-35 were analysed on a Waters UPLC H-Class with UV detection PDA equipped with an Acquity UPLC BEH C18 1.7 μm 2.1×50 mm column. MS spectra were recorded on a Waters SQ Detector 2 (electrospray) instrument, with the following gradient conditions:

Analytical method: (A: 0.1% HCOOH in H₂O, B: 0.1% HCOOH in ACN):

Purification of final products 14-30 was performed on a Waters Preparative LC (Autosampler 2707, Quaternary gradient module 2535, UV detector 2489, fraction collector WFCIII): column ACE 5 C18 250×21.2 mm², buffer: 0.1% TFA in H₂O, ACN; Flow 20 mL/min. Detection: 214 and 254 nm. Products were isolated as TFA salts.

Purification of final products 31-35 was performed on a Waters Preparative LC (Sample Manager 2767 (Fraction collector), Binary gradient module 2545, with two 515 HPLC pump and a System Fluidics Organiser SFO, Photodiode Array Detector 2998:column X Select CSH Prep C18 5 μm OBD 19×250 mm column, buffer: A: 0.1% HCOOH in H₂O, B: 0.1% HCOOH in CAN, Flow 20 mL/min. MS spectra were recorded on a Waters SQ Detector 2 (electrospray) instrument. Products were isolated as formate salts

NMR data was obtained on an Agilent 500 MHz or 700 MHz instrument in DMSO-d₆ with the chemical shifts referenced to solvent signals (DMSO-d₆, ¹H 2.50 ppm and ¹³C 39.52 ppm) relative to TMS. Peak multiplicities are designated by the following abbreviations: s, singlet; br s, broad singlet; d, doublet; t, triplet; q, quartet; p, pentet; m, multiplet; dd, doublet of doublets; ddd, doublet of doublet of doublets; td, triplet of doublets; tdd, triplet of doublets of doublets; tt, triplet of triplets.

HRMS spectra were recorded on an electrospray quadrupole time-of-flight Maxis 3G from Bruker using positive mode.

Allyl esters of amino acids were obtained from: Boc-L-Phe-OH, Boc-D-Phe-OH, Boc-L-Leu-OH, Boc-D-Leu-OH, Boc-L-Ala-OH, Boc-D-Ala-OH, Fmoc-L-Tyr-OH, Fmoc-L-Lys(Boc)-OH using standard esterification (allyl alcohol, EDC, DMAP, DCM) and deprotection methods: (50% TFA in DCM for Boc) or 20% piperidine in DMF (for Fmoc). Amino acids esters were isolated as free bases prior to resin loading.

### Results and Discussion

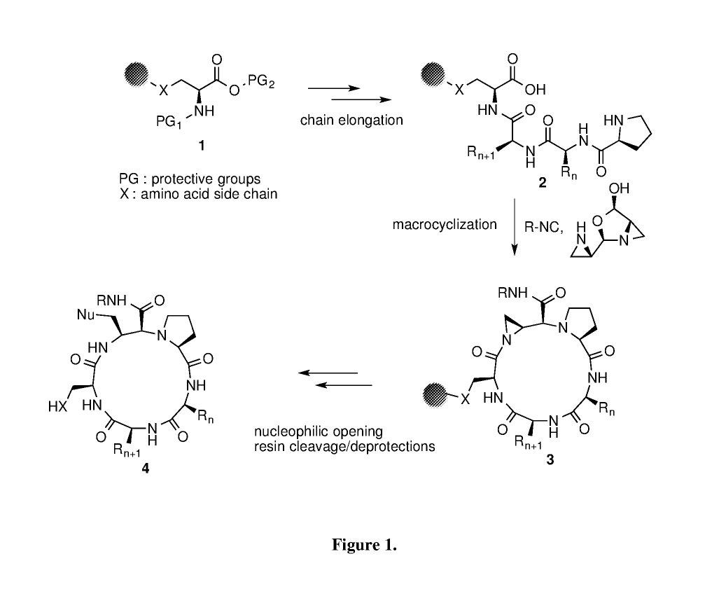

We sought a process in which every synthetic step is implemented on solid support, in order to maximize the possible generation of diversity. Key steps in our design (FIG. 1) included peptide elongation, pivotal three-component macrocyclization, subsequent nucleophilic ring-opening of the newly formed aziridine, and final resin cleavage with simultaneous deprotection of side chains. In order to address these requirements, we decided to attach the precursor peptide via the side chain of a suitably protected C-terminal amino acid. Accordingly (FIG. 1), we thought that a reasonable general strategy might involve side chain attachment of the amino acid, protected on the amino and the carboxylate positions, to deliver precursor 1. Subsequent chain elongation, ideally via standard Fmoc chemistry, followed by N- and C-terminal deprotections, would deliver linear precursor 2. The latter would then undergo macrocyclization in the presence of aziridine aldehyde dimer and isocyanide to produce macrocycle 3. Finally, we thought that the in situ nucleophilic opening of the newly formed acyl aziridine 3 would be followed by acid-mediated resin cleavage and concomitant side chain deprotection(s), to deliver the desired product 4, which are purified by reverse-phase HPLC. This synthetic approach offers several avenues for diversity via ring size, stereochemistry and side chains of amino acids, aziridine aldehyde dimer, and nucleophile. In this manuscript, we report a successful realization of this workflow and demonstrate its utility using a representative set of 9- to 18-membered rings bearing diverse amino acids and nucleophiles.

Several strategies were selected to attach different amino acids via their side chain to the solid support (Scheme 1). Fmoc-Glu-OA11 was attached directly to Wang resin via its side chain carboxylate in the presence of N,N′-diisopropylcarbodiimide (DIC) and 4-dimethylaminopyridine (DMAP) in DCM, to deliver precursor 5.[26] For the attachment of Ser and Thr, Merrifield resin was first functionalized to generate Ellman resin bearing a tetrahydropyranyl (THP) linker by reacting with 3,4-dihydro-2H-pyran-2-methanol and sodium hydride .[27] A subsequent reaction with the hydroxyl group of Fmoc-Ser-OAll or Fmoc-Thr-OAll in the presence of camphorsulfonic acid in DCM delivered precursors 6 and 7, respectively. [27] Anchoring of Lys via its side chain started with reaction of Wang resin with p-nitrophenyl chloroformate in the presence of pyridine in DCM, to generate the corresponding carbonate. Subsequent reaction with Fmoc-Lys-Oll in the presence of Hunig's base in DMF delivered precursor 8.[28] Finally, anchoring of Gln was prepared by amide bond formation between Fmoc-Glu-OAll and deprotected Rink amide resin in the presence of DIC, DIPEA, and DMAP in DCM, to deliver precursor 9. Generally, resin loadings were determined via Fmoc cleavage procedure and found to be 0.40 mmol/g for the DHP resins, or similar to the suppliers' loadings (0.68 mmol of Fmoc-L-GluOll/g, 0.66 mmol of Fmoc-L-LysOAll/g, 0.47 mmol of Fmoc-L-GlnOAll/g). Loadings were consistent with values obtained by cleavage in the presence of TFA:DCM 1:1. The purity of cleavage products was confirmed by LC-MS and found to be generally excellent (>95%, UV monitoring).

A typical synthesis is depicted in Scheme 2. Starting from Fmoc-Glu-OAll attached to Wang resin (Scheme 1), the Fmoc group was initially deprotected in the presence of 20% piperidine/DMF. Fmoc chemistry using HATU-mediated couplings was then used to assemble the final linear amino acid sequence. Deprotection of the allyl ester in the presence of palladium tetrakis(triphenylphosphine) and phenylsilane in DCM and Fmoc removal, delivered the macrocyclization precursor, H-(D)Pro-(D)Phe-Glu-OH 11, anchored on the resin via the Glu side chain [29, 30] The above reactions were driven to completion by using 3 eq. of reagents and were followed by UPLC-MS. Typically, macrocyclization precursors had a purity >90% as determined after mini-cleavage (2-5 mg resin in TFA:DCM 1:1) by UPLC-MS (UV monitoring).

The critical macrocyclization step was implemented in the form of a disrupted three-component Ugi reaction in the presence of aziridine aldehyde dimer 12 and t-butyl isocyanide. Previous experience with the solution-phase macrocyclization revealed that the reaction performs best in TFE as a solvent. However, TFE did not give satisfactory resin swelling, which is critical for reaction completion on solid support [31] A rapid screening of various solvents led us to choose DCM:TFE 1:1 as the best compromise between resin swelling and reactivity. Thus, macrocyclization was run in the presence of aziridine aldehyde dimer (1 eq. with respect to measured loading), t-butyl isocyanide (2 eq.) in DCM:TFE 1:1 at ambient temperature, to deliver acyl aziridine-containing macrocycle 13 (Scheme 2). The acyl aziridine was ring-opened in situ with thiophenol in the presence of DIPEA in DMF. Finally, the macrocycle was cleaved from the resin using 50% TFA in DCM. The crude material was collected by filtration and evaporation, and macrocycle 14 was isolated by MS-triggered preparative HPLC. This method was used to generate macrocycles 14-35 (Table 1).

A few observations can be made regarding this reaction sequence. First, the reaction performed well to deliver macrocycles of various ring sizes, ranging from 9 atoms (15, 16 derived from dipeptides) to 18 atoms in the ring (31-35 derived from pentapeptides). It is noteworthy that the yields for 9- and 12-membered rings were reasonable, knowing that these medium-sized rings are particularly difficult to close in the case of homodetic peptides.[32] Compared to homodetic cyclic tri- and tetrapeptides, which contain three or four amide bonds, respectively, the replacement of one amino acid by the substituted aminoethyl moiety originating from the three-component reaction, is expected to reduce ring strain via the removal of one amide bond. Second, macrocyclization proved tolerant of all families of side chain functionalities, including apolar and polar side chains bearing a protecting group. Third, the macrocyclization step was tolerant of variations in stereochemistry at every position, provided that the stereochemistry of the Pro residue on the N-terminus of the chain was matched to that of the aziridine aldehyde dimer (i.e., L-Pro reacts cooperatively with aziridine aldehyde dimer derived from L-Ser, while D-Pro matches the aziridine aldehyde dimer derived from D-Ser). Mismatched stereochemistry between the Pro residue and the dimer typically gave intractable mixtures as determined by UPLC-MS. This observation was rationalized recently by Zaretsky et al., who demonstrated in solution that, the match of the stereochemistry between the aziridine aldehyde dimer and amino acid partners was paramount to obtaining good diastereoselectivity with the disrupted Ugi reaction when using secondary amino acids. [33] Likewise, the introduction of non-alpha-amino acids in the chain was well tolerated (e.g., 24-26).

The aziridine ring of 3 (FIG. 1) was opened on resin with a variety of nucleophiles, exemplified here with different thiols (14, 19, 22, 23, 29, 30) and thiobenzoic acid (15). It should be noted, however, that all attempts to perform acid-mediated cleavage from the resin and isolation of aziridine-containing macrocycle 3 failed, which reflects the relative instability of the aziridine moiety.

As reported in Table 1, starting from 0.1 mmol resin (typically 160 mg of a 0.6 mmol/g nominal loading resin), macrocycles were isolated after synthesis, MS-triggered prep HPLC purification and lyophilization, in quantities ranging from 5 to 25 mg (19 and 24, respectively). These quantities correspond to isolated yields of 7-31% for the 7-, 9-, 11- or 13-step syntheses of ring systems built on di-, tri-, tetra- and pentapeptides, respectively. Hence, this is an efficient process overall.

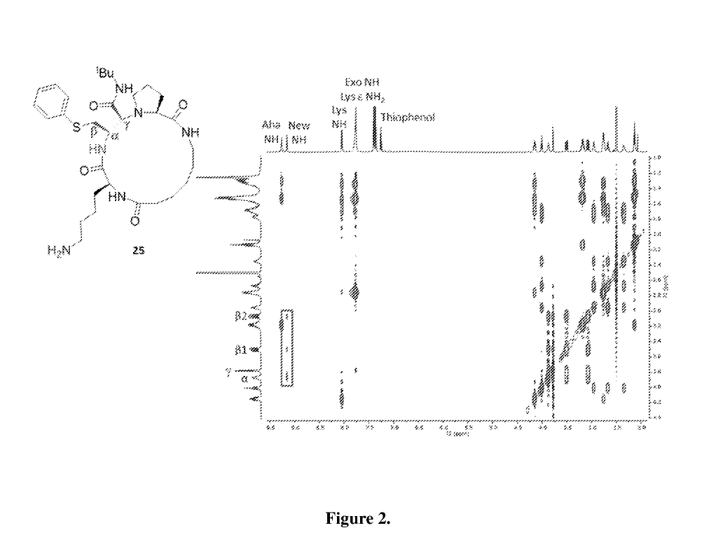

Once the purity of the peptides was verified by LC-MS, the macrocycles were analyzed using 1D ^{l}H and ¹³C NMR, as well as 2D ¹H-¹H and ¹H-¹³C NMR spectroscopic techniques. Successful macrocyclization was noted by the appearance of a new amide NH peak with 2D TOCSY crosspeaks to the adjacent linker region (FIG. 2). The structure of compound 25 has the linker atoms both annotated and highlighted in red. We observed the corresponding crosspeaks from the new NH amide to the alpha, beta, and gamma protons as shown on the TOCSY spectrum. The nucleophilic aziridine ring-opening was confirmed to proceed with the same regioselectivity as the solution-phase protocol, supported by NMR evidence of a downfield diastereotopic methylene at the beta position.

In conclusion, the reported methodology herein provides a versatile tool for the solid-phase parallel synthesis of diversified libraries of macrocyclic peptidomimetics. The reaction sequence is completely implemented on solid-phase, which minimizes transfers and user-intensive manipulations. The method was used to generate a library of several hundred macrocycles, 22 of which are reported herein. This was achieved using 24-well Mettler-Toledo parallel synthesis blocks.[34] Typically, the synthesis of a subset of 48 macrocycles is achievable in two weeks by a single chemist (excluding purification and lyophilization). Interestingly, the method is tolerant of a broad diversity in terms of ring size (9- to 18-membered rings) as well as the nature and stereochemistry of amino acids contained in the ring, providing molecules which also possess a non-peptidic exocyclic element as an additional point of diversity or subsequent functionalization. At a time when macrocycles are generating tremendous interest in the drug discovery community, we anticipate this method to become broadly applicable.

Compound Characterization

14

3-((3R,6S,9S,10R,14aR)-3-benzyl-10-(tert-butylcarbamoyl)-1,4,7-trioxo-9-((phenylthio)methy 1)tetradecahydropyrrolo[1,2-a][1,4,7,10]tetmazacyclododecin-6-yl)propanoic acid

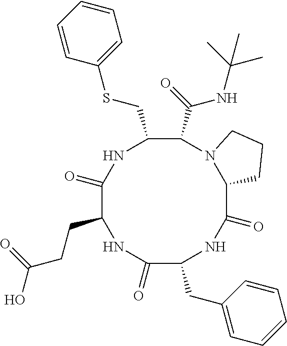 [SMILES: CC(C)(C)NC(=O)[C@H]1[C@@H](CSc2ccccc2)NC(=O)[C@H](CCC(=O)O)NC(=O)[C@@H](Cc2ccccc2)NC(=O)[C@H]2CCCN21]

¹H NMR: (700 MHz, DMSO-d₆) δ9.68 (d, J=8.8 Hz, 1H), 9.38 (s, 1H), 8.26 (d, J=8.0 Hz, 1H), 7.79 (s, 1H), 7.45-7.32 (m, 4H), 7.31-7.23 (m, 5H), 7.20 (m, 1H), 4.81 (td, J=9.3, 4.7 Hz, 1H), 4.37 (td, J=8.4, 4.9 Hz, 1H), 3.98 (t, J=8.5 Hz, 1H), 3.68 (m, 2H), 3.39-3.31 (m, 2H), 3.15-3.07 (m, 1H), 2.96-2.84 (m, 2H), 2.57 (m, 1H), 2.23 (q, J=10.5, 7.6 Hz, 3H), 2.09-2.00 (m, 1H), 1.85 (ddd, J=16.1, 14.2, 7.7 Hz, 1H), 1.61 (q, J=7.0, 5.2 Hz, 2H), 1.23 (s, 9H), 1.14 (dd, J=21.4, 11.5 Hz, 1H).

¹³C NMR: (126 MHz, DMSO-d₆) δ173.7, 172.5, 167.8, 167.2, 166.2, 136.6, 134.8, 129.3, 129.2, 128.7, 128.3, 126.8, 126.5, 58.3, 56.4, 55.7, 51.7, 51.5, 50.6, 50.5, 48.6, 36.6, 33.0, 31.1, 29.9, 28.2, 26.5, 23.1.

HRMS: [M+H]⁺ calculated for C₃₃H₄₄N₅O₆S: 638.3012; found: 638.3017.

LC-MS: t_{R} 8.34 min, 96% (UV, 214+254 nm); (MH⁺) 639.

15

3-((3S,6R,7S,11aS)-6-((benzoylthio)methyl)-7-(tert-butylcarbamoyl)-1,4-dioxodecahydro-1H-pyrrolo[1,2-a][1,4,7]triazonin-3-yl)propanoic acid

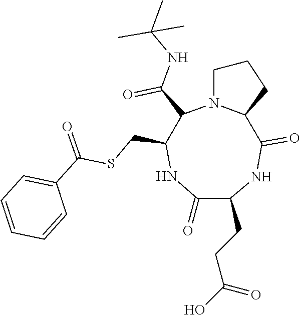

¹H NMR: (500 MHz, DMSO-d₆) δ9.55 (s, 1H), 9.07 (d, J=8.6 Hz, 1H), 7.97-7.89 (m, 2H), 7.80 (s, 1H), 7.76-7.67 (m, 1H), 7.65-7.52 (m, 2H), 4.56 (td, J=8.8, 4.7 Hz, 1H), 3.99 (q, J=5.8 Hz, 1H), 3.57 (dd, J=6.4, 3.4 Hz, 2H), 3.44 (dd, J=14.0, 5.3 Hz, 1H), 3.30-3.20 (m, 2H), 2.46-2.27 (m, 4H), 2.17 (dtd, J=13.9, 7.7, 4.6 Hz, 1H), 2.08-1.95 (m, 1H), 1.87 (q, J=7.7, 7.0 Hz, 1H), 1.84-1.69 (m, 2H), 1.30 (s, 9H).

¹³C NMR: ¹³C NMR (126 MHz, DMSO-d₆) δ190.6, 173.4, 170.4, 166.9, 165.6, 136.1, 134.2, 129.2, 127.0, 59.9, 55.7, 53.4, 52.4, 50.5, 50.0, 30.9, 29.8, 29.0, 28.3, 26.0, 23.1.

HRMS: [M+H]⁺ calculated for C₂₅H₃₅N₄O₆S: 519.2277; found: 519.2273.

LC-MS: t_{R} 9.67 min; 98% (UV, 214+254 nm); (MH⁺) 520.

16

(3S,6R,7S,11aS)-N-(tert-butyl)-3-(hydroxymethyl)-1,4-dioxo-6-((phenylthio)methyl)decahydro-1II-pyrrolo[1,2-a][1,4,7]triazonine-7-carboxamide

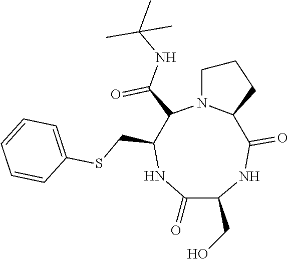

¹H NMR: (700 MHz, DMSO-d₆) δ9.33 (br s, 2H), 7.73 (s, 1H), 7.48-7.21 (m, 5H), 4.59 (d, J=6.1 Hz, 1H), 4.11 (t, J=8.1 Hz, 1H), 3.84-3.73 (m, 4H), 3.39-3.32 (m, 1H), 3.21 (dd, J=14.1, 7.2 Hz, 1H), 2.95 (d, J=9.0 Hz, 1H), 2.65 (d, J=7.3 Hz, 1H), 2.48-2.40 (m, 1H), 1.80-1.72 (m, 1H), 1.68 (p, J=8.5, 7.4 Hz, 2H), 1.25 (s, 9H).

¹³C NMR: (126 MHz, DMSO-d₆) δ169.1, 167.1, 166.0, 134.9, 129.3, 128.8, 126.5, 61.0, 58.7, 57.0, 55.4, 52.1, 50.5, 48.7, 33.2, 31.1, 28.3, 23.1.

HRMS: [M+H]⁺ calculated for C₂₂H₃₃N₄O₄S: 449.2223; found: 449.2217.

LC-MS: t_{R} 6.27 min, 94% (UV, 214+254 nm); (MH⁺) 639.

17

(3S,6S,9R,10S,14aS)-6-(3-amino-3-oxopropyl)-3-(4-aminobutyl)-N-(tert-butyl)-1,4,7-trioxo-9-((phenylthio)methyl)tetradecahydropyrrolo[1,2-a][1,4,7,10]tetraazacyclododecine-10-carboxamide

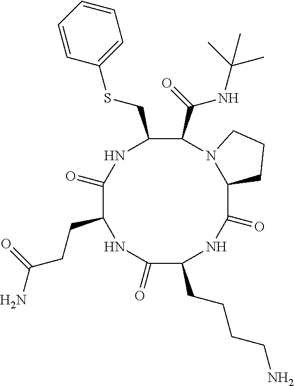 [SMILES: CC(C)(C)NC(=O)[C@@H]1[C@H](CSc2ccccc2)NC(=O)[C@H](CCC(N)=O)NC(=O)[C@H](CCCCN)NC(=O)[C@@H]2CCCN12]

¹H NMR: (500 MHz, DMSO-d₆) δ9.64 (d, J=8.3 Hz, 1H), 9.47 (s, 1H), 8.33 (d, J=7.9 Hz, 1H), 7.91 (s, 1H), 7.45-7.22 (m, 6H), 6.82 (s, 1H), 4.47 (td, J=8.5, 5.3 Hz, 1H), 4.28-4.20 (m, 1H), 4.05 (t, J=8.1 Hz, 1H), 3.93-3.82 (m, 2H), 3.44 (td, J=14.1, 5.0 Hz, 1H), 3.16-3.10 (m, 1H), 3.02-2.90 (m, 1H), 2.73 (dtd, J=30.2, 8.9, 5.3 Hz, 4H), 2.49-2.41 (m, 1H), 2.21-1.96 (m, 3H), 1.93-1.82 (m, 2H), 1.80-1.63 (m, 3H), 1.60-1.48 (m, 2H), 1.37-1.28 (m, 2H), 1.22 (s, 9H).

¹³C NMR: (126 MHz, DMSO-d₆) δ173.3, 172.8, 168.6, 167.4, 166.2, 134.8, 129.4, 128.8, 126.6, 57.9, 55.4, 54.6, 51.8, 51.6, 50.6, 48.2, 38.6, 33.0, 31.4, 31.3, 30.2, 28.2, 26.6, 26.6, 23.3, 22.1.

HRMS: [M+H]⁺ calculated for C₃₀H₄₈N₇O₅S: 618.3438; found: 618.3449.

LC-MS: t_{R} 5.22 min; 95% (UV, 214+254 nm); (MH⁺) 618.

18

(3R,6S,9R,10S,14aS)-6-(4-aminobutyl)-3 -benzyl-N-(tert-butyl)-1,4,7-trioxo-9-((phenylthio)methyl)tetradecahydropyrrolo[1,2-a][1,4,7,10]tetraazacyclododecine-10-carboxamide

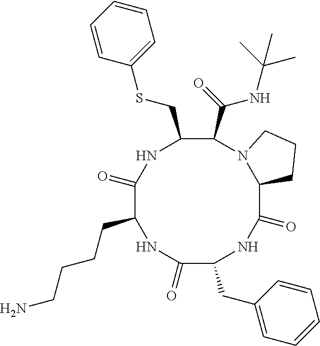 [SMILES: CC(C)(C)NC(=O)[C@@H]1[C@H](CSc2ccccc2)NC(=O)[C@H](CCCCN)NC(=O)[C@@H](Cc2ccccc2)NC(=O)[C@@H]2CCCN12]

¹H NMR: (700 MHz, DMSO-d₆) δ9.57 (d, J=8.7 Hz, 1H), 9.47 (s, 1H), 8.53 (d, J=7.7 Hz, 1H), 7.74 (s, 1H), 7.44-7.18 (m, 10H), 4.98-4.87 (m, 1H), 4.11 (ddd, J=9.0, 7.7, 4.7 Hz, 1H), 4.01 (t, J=8.5 Hz, 1H), 3.83 (q, J=6.3 Hz, 1H), 3.76 (d, J=4.2 Hz, 1H), 3.36 (dd, J=14.1, 6.2 Hz, 1H), 3.13 (ddd, J=27.3, 13.9, 7.6 Hz, 2H), 3.01 (dd, J=13.7, 7.2 Hz, 1H), 2.92 (d, J=7.2 Hz, 1H), 2.73-2.65 (m, 3H), 2.41-2.32 (m, 1H), 1.77-1.60 (m, 3H), 1.60-1.40 (m, 4H), 1.23 (s, 9H), 1.12-1.04 (m, 2H).

¹³C NMR: (126 MHz, DMSO-d₆) δ172.8, 168.1, 167.1, 165.5, 135.9, 134.9, 129.3, 129.2, 128.5, 128.3, 126.7, 126.4, 58.4, 55.7, 55.3, 51.9, 51.5, 50.5, 48.5, 36.5, 32.9, 31.1, 30.2, 28.2, 26.5, 23.1, 22.0.

HRMS: [M+2H]²⁺ calculated for C₃₄H₅₀N₆O₄S: 319.1802; found: 319.1812.

LC-MS: t_{R} 7.60 min; 90% (UV, 214+254 nm); (MH⁺) 637.

19

(3S,6S,9R,10S,14aS)-6-(3-amino-3-oxopropyl)-N-(tert-butyl)-9-(((3-fluorophenyl)thio)methyl)-3-isobutyl-1,4,7-trioxotetradecahydropyrrolo[1,2-a][1,4,7,10]tetraazacyclododecine-10-carboxamide

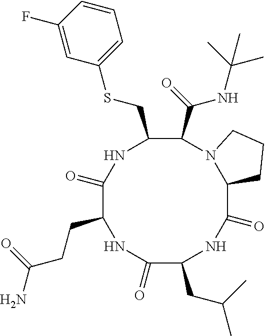 [SMILES: CC(C)C[C@@H]1NC(=O)[C@@H]2CCCN2[C@H](C(=O)NC(C)(C)C)[C@H](CSc2cccc(F)c2)NC(=O)[C@H](CCC(N)=O)NC1=O]

¹H NMR: (500 MHz, DMSO-d₆) δ9.67 (d, J=8.3 Hz, 1H), 9.55 (s, 1H), 8.26 (d, J=8.0 Hz, 1H), 7.90 (s, 1H), 7.40 (td, J=8.2, 6.4 Hz, 1H), 7.29 (s, 1H), 7.27-7.17 (m, 2H), 7.13-7.04 (m, 1H), 6.84-6.79 (m, 1H), 4.48 (q, J=7.6 Hz, 1H), 4.24 (ddd, J=9.3, 8.0, 5.0 Hz, 1H), 4.05 (t, J=8.3 Hz, 1H), 3.95 (dt, J=11.4, 5.6 Hz, 1H), 3.84 (d, J=4.2 Hz, 1H), 3.43 (dd, J=13.7, 5.9 Hz, 1H), 3.20 (dd, J=14.3, 8.0 Hz, 1H), 2.95 (d, J=7.8 Hz, 1H), 2.74 (q, J=8.6 Hz, 1H), 2.48-2.40 (m, 1H), 2.16 (dd, J=8.7, 7.1 Hz, 2H), 2.11-2.01 (m, 1H), 1.95-1.83 (m, 1H), 1.81-1.49 (m, 6H), 1.23 (s, 9H), 0.90 (d, J=6.5 Hz, 3H), 0.85 (d, J=6.5 Hz, 3H).

¹³C NMR: (126 MHz, DMSO-d₆) δ173.2, 172.8, 167.4, 163.4, 161.4, 137.7, 131.0, 124.2, 114.8, 113.1, 104.6, 58.0, 55.5, 53.4, 51.8, 50.6, 50.5, 48.3, 39.1, 32.7, 31.4, 31.3, 28.2, 26.6, 24.2, 23.3, 22.8, 21.2.

HRMS: [M+H]⁺ calculated for C₃₀H₄₆FN₆O₅S: 621.3234; found: 621.3229.

LC-MS: t_{R} 6.87 min; 93% (UV, 214+254 nm); (MH⁺) 621.

20

(3S,6S,9R,10S,14aS)-6-(3-amino-3-oxopropyl)-N-(tert-butyl)-9-(((3-fluorophenyl)thio)methyl)-3-isobutyl-1,4,7-trioxotetmdecahydropyrrolo[1,2-a][1,4,7,10]tetraazacyclododecine-10-carboxamide

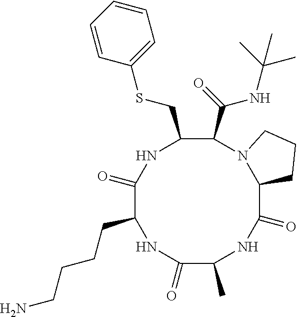 [SMILES: C[C@@H]1NC(=O)[C@@H]2CCCN2[C@H](C(=O)NC(C)(C)C)[C@H](CSc2ccccc2)NC(=O)[C@H](CCCCN)NC1=O]

¹H NMR: (500 MHz, DMSO-d₆) δ9.63 (d, J=8.2 Hz, 1H), 9.36 (s, 1H), 8.11 (d, J=7.9 Hz, 1H), 7.90 (s, 1H), 7.46-7.33 (m, 4H), 7.31-7.23 (m, 1H), 4.61 (p, J=7.1 Hz, 1H), 4.21 (ddd, J=9.3, 7.9, 5.1 Hz, 1H), 4.03 (t, J=8.1 Hz, 1H), 3.89-3.85 (m, 2H), 3.49 (dd, J=14.2, 4.6 Hz, 1H), 3.16-3.06 (m, 1H), 2.94 (d, J=8.0 Hz, 1H), 2.81-2.65 (m, 3H), 2.47-2.35 (m, 1H), 1.88-1.61 (m, 5H), 1.59-1.48 (m, 2H), 1.44-1.31 (m, 5H), 1.23 (s, 9H).

¹³C NMR: (126 MHz, DMSO-d₆) δ172.9, 169.3, 167.4, 165.8, 134.8, 129.4, 128.7, 126.5, 57.8, 55.3, 52.1, 51.8, 50.6, 50.3, 48.1, 38.6, 32.8, 31.4, 30.0, 28.2, 26.6, 23.4, 22.6, 17.1.

HRMS: [M+H]⁺ calculated for C₂₈H₄₅N₆O₄S: 561.3223; found: 561.3227.

LC-MS: t_{R} 4.67 min; 97% (UV, 214+254 nm); (MH⁺) 561.

21

(3R,6S,9R,10S,14aS)-N-(tert-butyl)-6-(hydroxymethyl)-3-methyl-1,4,7-trioxo-9-((phenylthio)methyl)tetradecahydropyrrolo[1,2-a][1,4,7,10]tetraazacyclododecine-10-carboxamide

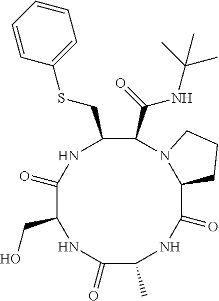 [SMILES: C[C@H]1NC(=O)[C@@H]2CCCN2[C@H](C(=O)NC(C)(C)C)[C@H](CSc2ccccc2)NC(=O)[C@H](CO)NC1=O]

¹H NMR: 500 MHz, DMSO-d₆) δ9.36 (d, J=8.3 Hz, 1H), 9.14 (s, 1H), 8.24 (d, J=7.8 Hz, 1H), 7.77 (s, 1H), 7.45-7.33 (m, 4H), 7.30-7.23 (m, 1H), 4.63-4.54 (m, 1H), 4.30 (ddd, J=7.9, 5.2, 4.1 Hz, 1H), 4.04 (t, J=8.2 Hz, 1H), 3.85 (td, J=6.9, 4.1 Hz, 1H), 3.78 (d, J=4.3 Hz, 1H), 3.74 (dd, J=11.0, 5.2 Hz, 1H), 3.66 (dd, J=10.9, 4.1 Hz, 1H), 3.50-3.40 (m, 1H), 3.14 (dd, J=14.0, 7.7 Hz, 1H), 2.95 (s, 1H), 2.70-2.61 (m, 1H), 2.43-2.33 (m, 1H), 1.83-1.63 (m, 3H), 1.36 (d, J=6.8 Hz, 3H), 1.25 (s, 9H).

¹³C NMR: (126 MHz, DMSO-d₆) δ171.5, 169.3, 167.2, 165.2, 134.9, 129.4, 128.6, 126.5, 61.0, 58.6, 55.3, 55.0, 52.0, 50.5, 50.2, 48.5, 33.0, 31.1, 28.2, 23.2, 17.1.

HRMS: [M+H]⁺ calculated for C₂₅H₃₈N₅O₅S: 520.2594; found: 520.2591.

LC-MS: t_{R} 6.09 min; 96% (UV, 214+254 nm); (MH⁺) 520.

22

(3 S,6S,9R,10S,14aS)-6-(4-aminobutyl)-3-benzyl-N-(tert-butyl)-9-(((2,4-difluorophenyl)thio)methyl)-1,4,7-trioxotetradecahydropyrrolo[1,2-a][1,4,7,10]tetraazacyclododecine-10-carboxamide

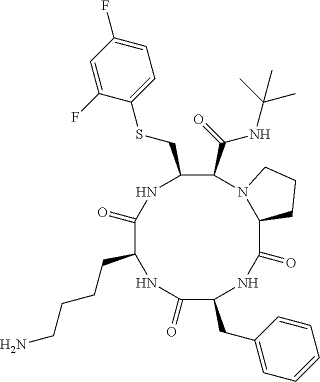 [SMILES: CC(C)(C)NC(=O)[C@@H]1[C@H](CSc2ccc(F)cc2F)NC(=O)[C@H](CCCCN)NC(=O)[C@H](Cc2ccccc2)NC(=O)[C@@H]2CCCN12]

¹H NMR: (700 MHz, DMSO-d₆) δ9.82 (d, J=8.9 Hz, 1H), 9.22 (s, 1H), 8.24 (d, J=8.1 Hz, 1H), 7.83 (s, 1H), 7.60 (td, J=8.7, 6.3 Hz, 1H), 7.38 (td, J=9.4, 2.7 Hz, 1H), 7.28 (d, J=4.4 Hz, 4H), 7.21 (q, J=4.5 Hz, 1H), 7.17-7.11 (m, 1H), 4.84 (td, J=9.7, 4.1 Hz, 1H), 4.25 (ddd, J=9.3, 8.0, 5.1 Hz, 1H), 3.98 (t, J=8.5 Hz, 1H), 3.76 (d, J=4.1 Hz, 1H), 3.65 (t, J=6.9 Hz, 1H), 3.35-3.30 (m, 2H), 3.00 (dd, J=14.1, 8.5 Hz, 1H), 2.90 (ddd, J=13.8, 11.7, 7.6 Hz, 2H), 2.76 (dq, J=13.6, 7.2, 6.4 Hz, 2H), 2.58-2.53 (m, 1H), 2.23 (t, J=6.1 Hz, 1H), 1.87-1.78 (m, 1H), 1.73 (dtd, J=14.1, 9.5, 4.9 Hz, 1H), 1.65-1.59 (m, 2H), 1.58-1.51 (m, 2H), 1.47-1.30 (m, 2H), 1.21 (s, 9H).

¹³C NMR: (126 MHz, DMSO-d₆) δ172.9, 168.1, 167.3, 166.2, 136.7, 134.7, 129.2, 128.3, 126.8, 116.7, 112.6, 105.0, 57.8, 56.1, 55.5, 52.1, 51.7, 50.5, 48.4, 38.6, 36.5, 33.6, 31.5, 30.2, 28.1, 26.6, 23.3, 22.5.

HRMS: M+2H]²⁺ calculated for C₃₄H₄₈F₂N₆O₄S: 337.1708; found: 337.1718.

LC-MS: t_{R} 6.25 min; 95% (UV, 214+254 nm); (MH⁺) 673.

23

(3S,6S,9R,10S,14aS)-6-(3-amino-3-oxopropyl)-N-(tert-butyl)-3-isobutyl-1,4,7-trioxo-9-((thiophen-2-ylthio)methyl)tetradecahydropyrrolo[1,2-a][1,4,7,10]tetraazacyclododecine-10-carboxamide

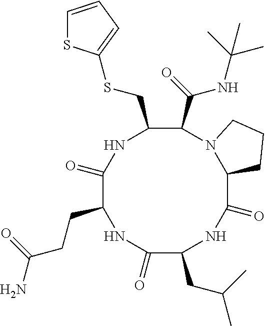 [SMILES: CC(C)C[C@@H]1NC(=O)[C@@H]2CCCN2[C@H](C(=O)NC(C)(C)C)[C@H](CSc2cccs2)NC(=O)[C@H](CCC(N)=O)NC1=O]

¹H NMR: (700 MHz, DMSO-d₆) δ9.65 (d, J=8.3 Hz, 1H), 9.39 (s, 1H), 8.20 (s, 1H), 7.88 (s, 1H), 7.71 (dd, J=5.4, 1.3 Hz, 1H), 7.32 (dd, J=3.5, 1.3 Hz, 1H), 7.24-7.19 (m, 1H), 7.10 (dd, J=5.3, 3.5 Hz, 1H), 6.80 (s, 1H), 4.47 (q, J=7.9 Hz, 1H), 4.23 (ddd, J=9.4, 8.0, 5.0 Hz, 1H), 4.04 (t, J=8.5 Hz, 1H), 3.89 (q, J=7.3, 5.7 Hz, 2H), 3.23 (dd, J=13.9, 5.7 Hz, 1H), 2.98 (t, J=10.7 Hz, 2H), 2.74 (td, J=9.1, 6.0 Hz, 1H), 2.47-2.40 (m, 1H), 2.14 (dd, J=8.7, 7.2 Hz, 2H), 2.09-2.02 (m, 1H), 1.91-1.81 (m, 1H), 1.79 (qt, J=7.3, 4.0 Hz, 1H), 1.75-1.65 (m, 3H), 1.65-1.53 (m, 2H), 1.21 (s, 9H), 0.90 (d, J=6.6 Hz, 3H), 0.84 (d, J=6.6 Hz, 3H).

¹³C NMR: (126 MHz, DMSO-d₆) δ173.3, 172.8, 169.0, 167.3, 166.4, 134.9, 131.8, 131.3, 128.3, 57.6, 55.5, 53.3, 51.8, 51.7, 50.6, 48.1, 39.0, 38.2, 31.5, 31.3, 28.2, 26.6, 24.3, 23.3, 22.8, 21.2.

HRMS: [M+H]⁺ calculated for C₂₈H₄₅N₆O₅S₂: 609.2885.

LC-MS: t_{R} 6.95 min; 90% (UV, 214+254 nm); (MH⁺) 609.

24

(7S,10R,11S,15aS)-7-(4-aminobutyl)-N-(tert-butyl)-1,5,8-trioxo-10-((phenylthio)methyl)tetradecahydro-1H-pyrrolo[2,1-c][1,4,7,10]tetraazacyclotridecine-11-carboxamide

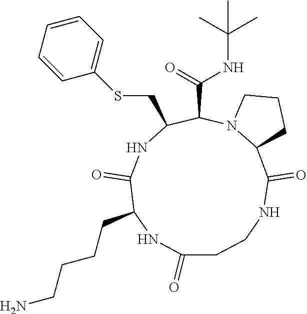 [SMILES: CC(C)(C)NC(=O)[C@@H]1[C@H](CSc2ccccc2)NC(=O)[C@H](CCCCN)NC(=O)CCNC(=O)[C@@H]2CCCN12]

¹H NMR: (500 MHz, DMSO-d₆) δ9.53-9.30 (m, 2H), 8.35 (d, J=7.8 Hz, 1H), 7.84 (s, 1H), 7.45-7.39 (m, 2H), 7.36 (dd, J=8.5, 7.0 Hz, 2H), 7.25 (ddt, J=7.8, 6.7, 1.2 Hz, 1H), 4.19 (ddd, J=9.0, 7.8, 5.0 Hz, 1H), 4.02 (t, J=8.2 Hz, 1H), 3.86 (q, J=6.9 Hz, 1H), 3.82 (d, J=4.3 Hz, 1H), 3.48 (ddd, J=14.1, 11.2, 6.5 Hz, 2H), 3.43-3.34 (m, 1H), 3.10 (dd, J=14.1, 7.4 Hz, 1H), 2.96 (ddd, J=9.1, 6.4, 3.0 Hz, 1H), 2.75 (td, J=7.5, 3.2 Hz, 2H), 2.67 (td, J=8.9, 6.2 Hz, 1H), 2.55-2.50 (m, 2H), 2.37-2.28 (m, 1H), 1.81-1.48 (m, 7H), 1.40-1.29 (m, 2H), 1.25 (s, 9H).

¹³C NMR: (126 MHz, DMSO-d₆) δ173.4, 169.9, 167.1, 165.1, 135.0, 129.3, 128.7, 126.5, 58.9, 55.2, 52.0, 51.9, 50.6, 48.9, 38.6, 37.8, 33.1, 32.9, 30.7, 30.6, 28.3, 26.7, 23.1, 22.5.

HRMS: [M+H]⁺ calculated for C₂₈H₄₄N₆O₄S: 561.3245; found: 561.3245.

LC-MS: t_{R} 4.77 min; 99% (UV, 214+254 nm); (MH⁺) 561.

25

(10S,13R,14S,18aS)-10-(4-aminobutyl)-N-(tert-butyl)-1,8,11-trioxo-13-((phenylthio)methyl)octadecahydropyrrolo[2,1-c] [1,4,7,10]tetraazacyclohexadecine-14-carboxamide

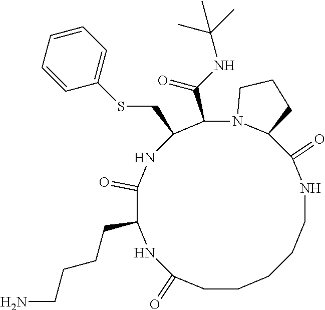 [SMILES: CC(C)(C)NC(=O)[C@@H]1[C@H](CSc2ccccc2)NC(=O)[C@H](CCCCN)NC(=O)CCCCCNC(=O)[C@@H]2CCCN12]

¹H NMR: (500 MHz, DMSO-d₆) δ9.26 (t, J=5.5 Hz, 1H), 9.16 (s, 1H), 8.04 (d, J=7.8 Hz, 1H), 7.76 (s, 1H), 7.45-7.33 (m, 4H), 7.29-7.22 (m, 1H), 4.15 (ddd, J=9.2, 7.7, 5.0 Hz, 1H), 4.01 (t, J=8.1 Hz, 1H), 3.87 (q, J=6.4, 5.5 Hz, 1H), 3.78 (d, J=4.3 Hz, 1H), 3.50 (dd, J=14.1, 6.3 Hz, 1H), 3.18 (dp, J=13.8, 6.5 Hz, 2H), 3.07 (dd, J=14.1, 7.8 Hz, 1H), 2.95 (ddd, J=9.1, 6.6, 3.1 Hz, 1H), 2.76 (q, J=6.2 Hz, 2H), 2.67 (td, J=8.9, 6.2 Hz, 1H), 2.34 (dt, J=11.7, 7.4 Hz, 1H), 2.13 (t, J=7.4 Hz, 2H), 1.70 (dddt, J=21.8, 18.6, 10.4, 4.1 Hz, 4H), 1.61-1.45 (m, 6H), 1.25 (s, 12H).

¹³C NMR: (126 MHz, DMSO-d₆) δ173.7, 172.2, 167.1, 164.8, 135.1, 129.3, 128.5, 126.4, 59.2, 55.2, 52.0, 51.6, 50.5, 49.1, 41.4, 38.6, 34.8, 32.9, 30.7, 30.5, 28.3, 26.6, 25.8, 24.8, 23.2, 22.5.

HRMS: [M+2H]²⁺ calculated for C₃₁H₅₁N₆O₄S: 302.1880; found: 302.1879.

LC-MS: t_{R} 5.12 min; 98% (UV, 214+254 nm); (MH⁺) 603.

26

(10S,13R,14S,18aS)-10-(4-aminobutyl)-N-(tert-butyl)-1,8,11-trioxo-13-((phenylthio)methyl)octadecahydropyrrolo[2,1-c][1,4,7,10]tetraazacyclohexadecine-14-carboxamide

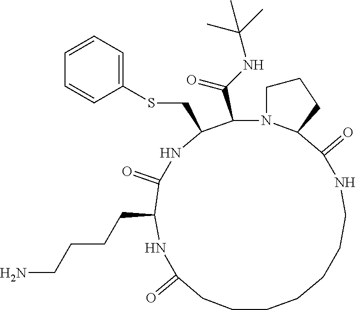 [SMILES: CC(C)(C)NC(=O)[C@@H]1[C@H](CSc2ccccc2)NC(=O)[C@H](CCCCN)NC(=O)CCCCCCCNC(=O)[C@@H]2CCCN12]

¹H NMR: (500 MHz, DMSO-d₆) δ9.28 (s, 1H), 9.17 (s, 1H), 8.02 (d, J=7.8 Hz, 1H), 7.77 (s, 1H), 7.45-7.32 (m, 4H), 7.30-7.21 (m, 1H), 4.14 (ddd, J=9.3, 7.8, 5.0 Hz, 1H), 4.02 (t, J=8.1 Hz, 1H), 3.93-3.83 (m, 1H), 3.79 (d, J=4.3 Hz, 1H), 3.51 (dd, J=14.1, 6.3 Hz, 1H), 3.19 (q, J=6.4 Hz, 2H), 3.07 (dd, J=14.1, 7.8 Hz, 1H), 2.96 (td, J=6.7, 3.3 Hz, 1H), 2.81-2.72 (m, 2H), 2.71-2.64 (m, 1H), 2.40-2.31 (m, 1H), 2.11 (t, J=7.5 Hz, 2H), 1.82-1.62 (m, 4H), 1.59-1.44 (m, 7H), 1.39-1.20 (m, 15H).

¹³C NMR: (126 MHz, DMSO-d₆) δ173.7, 172.4, 167.2, 164.9, 135.1, 129.3, 128.5, 126.4, 59.1, 55.3, 52.0, 51.6, 50.5, 49.1, 41.5, 38.6, 35.0, 32.9, 30.7, 30.5, 28.6, 28.3, 27.3, 26.6, 26.0, 25.2, 23.2, 22.5.

HRMS: [M+2H]²⁺ cultured for C₃₃H₅₅N₆O₄S: 316.2037; found: 316.2040.

LC-MS: t_{R} 5.59 min; 91% (UV, 214+254 nm); (MH⁺) 631.

27

3-((3R,6S,9S,12R,13S,17aS)-6-(4-aminobutyl)-13-(tert-butylcarbamoyl)-3-isobutyl-1,4,7,10-tetraoxo-12-((phenylthio)methyl)hexadecahydro-1H-pyrrolo[1,2-a][1,4,7,10,13]pentaazacyclopentadecin-9-yl)propanoic acid

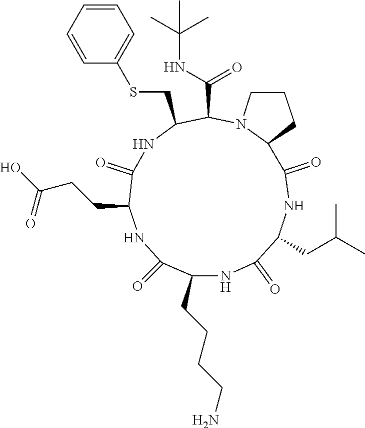 [SMILES: CC(C)C[C@H]1NC(=O)[C@@H]2CCCN2[C@H](C(=O)NC(C)(C)C)[C@H](CSc2ccccc2)NC(=O)[C@H](CCC(=O)O)NC(=O)[C@H](CCCCN)NC1=O]

¹H NMR: (700 MHz, DMSO-d₆) δ9.42 (d, J=8.8 Hz, 1H), 9.16 (s, 1H), 8.44 (d, J=7.6 Hz, 1H), 8.25 (d, J=8.0 Hz, 1H), 7.71 (s, 1H), 7.45-7.38 (m, 2H), 7.34 (dd, J=8.2, 7.4 Hz, 2H), 7.28-7.20 (m, 1H), 4.65-4.57 (m, 1H), 4.28-4.17 (m, 2H), 4.05 (t, J=8.4 Hz, 1H), 3.90 (dt, J=9.6, 4.9 Hz, 1H), 3.82 (d, J=4.1 Hz, 1H), 3.61 (dd, J=13.9, 5.6 Hz, 1H), 3.08 (dd, J=13.8, 8.8 Hz, 1H), 2.95 (t, J=7.7 Hz, 1H), 2.75 (m, 2H), 2.66 (td, J=9.4, 6.4 Hz, 1H), 2.42-2.35 (m, 1H), 2.27-2.14 (m, 2H), 2.00-1.92 (m, 1H), 1.80-1.72 (m, 2H), 1.72-1.50 (m, 8H), 1.41-1.29 (m, 2H), 1.23 (s, 9H), 0.94-0.83 (m, 6H).

¹³C NMR: (126 MHz, DMSO-d₆) δ173.7, 173.0, 171.2, 169.3, 167.3, 165.9, 134.9, 129.3, 128.6, 126.4, 57.9, 55.4, 53.3, 52.7, 51.9, 51.1, 50.5, 47.8, 39.8, 38.6, 32.9, 31.7, 31.3, 29.9, 28.2, 26.6, 26.4, 24.1, 23.4, 22.8, 22.1, 21.3.

HRMS: [M+H]⁺ calculated for C₃₆H₅₇N₇O₇S: 732.4140; found: 732.4138.

LC-MS: t_{R} 6.90 min; 91% (UV, 214+254 nm); (MH⁺) 732.

28

3-((3S,6S,9S,12S,13R,17aR)-6-benzyl-13-(tert-butylcarbamoyl)-3-isobutyl-1,4,7,10-tetraoxo-12-((phenylthio)methyl)hexadecahydro-1H-pyrrolo[1,2-a][1,4,7,10,13]pentaazacyclopentadecin-9-yl)propanoic acid

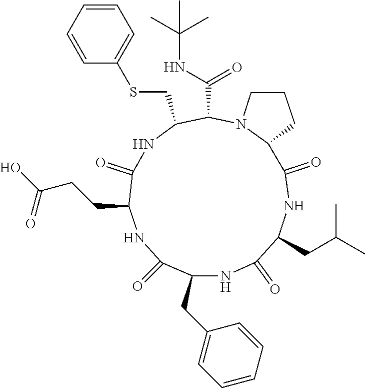 [SMILES: CC(C)C[C@@H]1NC(=O)[C@H]2CCCN2[C@@H](C(=O)NC(C)(C)C)[C@@H](CSc2ccccc2)NC(=O)[C@H](CCC(=O)O)NC(=O)[C@H](Cc2ccccc2)NC1=O]

¹H NMR: (500 MHz, DMSO-d₆) δ9.39 (d, J=9.1 Hz, 1H), 8.78 (s, 1H), 8.48 (d, J=7.9 Hz, 1H), 8.28 (d, J=8.7 Hz, 1H), 7.73 (s, 1H), 7.51-7.38 (m, 4H), 7.34-7.26 (m, 1H), 7.22-7.16 (m, 2H), 7.11-7.01 (m, 3H), 4.69 (ddd, J=10.2, 8.7, 4.1 Hz, 1H), 4.44-4.33 (m, 1H), 4.27 (ddd, J=9.2, 7.8, 5.0 Hz, 1H), 3.93 (t, J=8.6 Hz, 1H), 3.82 (d, J=4.0 Hz, 1H), 3.79-3.74 (m, 1H), 3.38 (dd, J=14.0, 5.8 Hz, 1H), 3.19-3.13 (m, 1H), 3.08 (dd, J=14.0, 4.0 Hz, 1H), 2.96 (t, J=7.8 Hz, 1H), 2.74 (dd, J=14.0, 10.2 Hz, 1H), 2.58 (q, J=8.9 Hz, 1H), 2.28 (ddd, J=8.6, 6.7, 4.5 Hz, 2H), 2.21 (td, J=13.5, 11.5, 6.7 Hz, 1H), 2.06-1.95 (m, 1H), 1.86-1.69 (m, 2H), 1.69-1.52 (m, 3H), 1.49 (dd, J=10.1, 5.4 Hz, 1H), 1.23 (s, 9H), 1.08 (p, J=10.5 Hz, 1H), 0.87 (d, J=6.2 Hz, 3H), 0.80 (d, J=6.1 Hz, 3H).

¹³C NMR: (126 MHz, DMSO-d₆) δ174.1, 173.4, 171.4, 168.8, 167.6, 166.0, 137.8, 135.1, 129.9, 129.7, 129.4, 128.2, 127.3, 126.6, 58.3, 55.7, 53.9, 53.2, 52.3, 51.6, 50.9, 48.3, 39.9, 38.2, 33.7, 32.0, 30.4, 28.6, 26.8, 24.3, 23.7, 23.2, 21.8.

HRMS: [M+H]⁺ calculated for C₃₉H₅₅N₆O₇S: 751.3847; found: 751.3856.

LC-MS: t_{R} 9.02 min; 95% (UV, 214+254 nm); (MH³⁰ ) 752.

29

(3R,6R,9S,12R,13S,17aS)-9-(4-aminobutyl)-6-benzyl-N-(tert-butyl)-12-(((2,4-dimethylphenyl)thio)methyl)-3-isobutyl-1,4,7,10-tetraoxohexadecahydro-1H-pyrrolo[1,2-a][1,4,7,10,13]pentaazacyclopentadecine-13-carboxamide

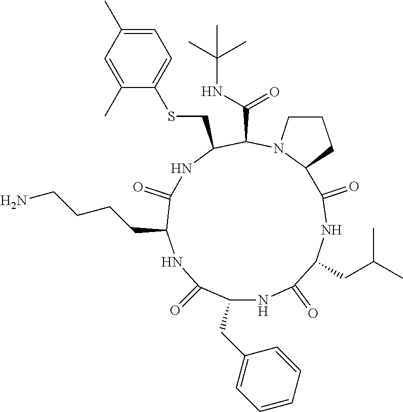 [SMILES: Cc1ccc(SC[C@@H]2NC(=O)[C@H](CCCCN)NC(=O)[C@@H](Cc3ccccc3)NC(=O)[C@@H](CC(C)C)NC(=O)[C@@H]3CCCN3[C@@H]2C(=O)NC(C)(C)C)c(C)c1]

¹H NMR: (500 MHz, DMSO-d₆) δ9.42 (d, J=9.1 Hz, 1H), 8.79 (s, 1H), 8.48 (d, J=8.0 Hz, 1H), 8.36 (d, J=8.9 Hz, 1H), 7.72 (s, 1H), 7.35 (d, J=7.9 Hz, 1H), 7.28 (s, 1H), 7.21-7.11 (m, 4H), 7.11-7.00 (m, 4H), 4.75 (td, J=9.2, 5.3 Hz, 1H), 4.48-4.37 (m, 1H), 4.16 (td, J=8.6, 4.9 Hz, 1H), 3.95 (t, J=8.6 Hz, 1H), 3.84 (d, J=3.9 Hz, 1H), 3.72 (q, J=6.4, 4.8 Hz, 1H), 3.29 (dd, J=13.8, 5.9 Hz, 1H), 3.06 (dd, J=13.8, 8.0 Hz, 1H), 3.02-2.94 (m, 2H), 2.73 (dd, J=13.1, 9.5 Hz, 3H), 2.65-2.56 (m, 1H), 2.37 (s, 3H), 2.22 (m, 4H), 1.78-1.42 (m, 9H), 1.23 (m, 11H), 1.16-1.04 (m, 1H), 0.86 (d, J=6.3 Hz, 3H), 0.81 (d, J=6.3 Hz, 3H).

¹³C NMR: (126 MHz, DMSO-d₆) δ173.7, 171.0, 168.8, 167.6, 166.1, 138.4, 137.7, 137.0, 131.8, 130.4, 130.1, 129.6, 128.1, 127.8, 126.5, 58.3, 55.7, 53.9, 53.2, 52.3, 52.0, 50.9, 48.1, 40.0, 39.0, 39.0, 33.6, 32.1, 31.1, 28.6, 27.0, 24.3, 23.8, 23.2, 22.6, 21.8, 20.9, 20.4.

HRMS: [M+2H]²⁺ calculated for C₄₂H₆₅N₇O₅S: 389.7379; found: 389.7378.

LC-MS: t_{R} 7.20 min; 94% (UV, 214+254 nm); (MH⁺) 778.

30

(3R,9S,12R,13S,17aS)-9-(4-aminobutyl)-N-(tert-butyl)-12-(((3,5-dichlorophenyl)thio)methyl)-3-isobutyl-1,4,7,10-tetraoxohexadecahydro-1H-pyrrolo[1,2-a][1,4,7,10,13]pentaazacyclopentadecine-13-carboxamide

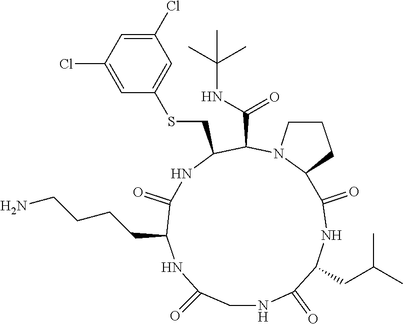 [SMILES: CC(C)C[C@H]1NC(=O)[C@@H]2CCCN2[C@H](C(=O)NC(C)(C)C)[C@H](CSc2cc(Cl)cc(Cl)c2)NC(=O)[C@H](CCCCN)NC(=O)CNC1=O]

¹H NMR: (500 MHz, DMSO-d₆) δ9.47 (d, J=8.7 Hz, 1H), 9.34 (s, 1H), 8.35 (t, J=5.6 Hz, 1H), 8.22 (d, J=7.8 Hz, 1H), 7.71 (s, 1H), 7.48 (t, J=1.8 Hz, 1H), 7.42 (d, J=1.9 Hz, 2H), 4.55 (t, J=8.5 Hz, 1H), 4.19 (td, J=8.4, 4.9 Hz, 1H), 4.07 (t, J=8.2 Hz, 1H), 3.95 (q, J=6.2 Hz, 1H), 3.87 (dd, J=16.7, 6.0 Hz, 1H), 3.74 (dd, J=16.2, 4.8 Hz, 2H), 3.53 (dd, J=14.2, 6.3 Hz, 1H), 3.21 (dd, J=14.1, 7.4 Hz, 1H), 2.96 (s, 1H), 2.78 (ddq, J=36.7, 16.3, 6.7 Hz, 5H), 2.38 (q, J=5.9 Hz, 6H), 1.86-1.47 (m, 9H), 1.40-1.29 (m, 2H), 1.24 (s, 9H), 0.89 (d, J=5.3 Hz, 3H), 0.84 (d, J=5.1 Hz, 3H).

¹³C NMR: (126 MHz, DMSO-d₆) δ173.3, 169.5, 168.3, 167.2, 166.1, 139.9, 134.7, 126.0, 125.8, 58.5, 55.7, 53.5, 51.9, 51.7, 50.5, 48.1, 41.8, 39.9, 38.6, 34.2, 32.8, 31.5, 30.7, 28.2, 26.6, 24.4, 24.0, 23.4, 23.0, 22.3, 21.1.

HRMS: [M+H]⁺ calculated for C₃₃H₅₂Cl₂N₇O₅S: 728.3149; found: 728.3162.

LC-MS: t_{R} 7.05 min; 95% (UV, 214+254 nm); (MH⁺) 728.

31

(3S,6S,9R,12S,15R,16S,20aS)-12-(4-aminobutyl)-N-(tert-butyl)-6,9-diisobutyl-3-methyl-1,4,7,10,13-pentaoxo-15-((phenylthio)methyl)icosahydropyrrolo[1,2-a][1,4,7,10,13,16]hexaazacyclooctadecine-16-carboxamide

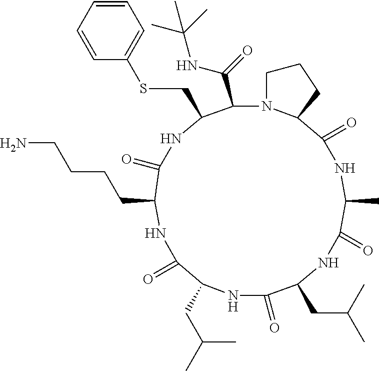 [SMILES: CC(C)C[C@@H]1NC(=O)[C@H](C)NC(=O)[C@@H]2CCCN2[C@H](C(=O)NC(C)(C)C)[C@H](CSc2ccccc2)NC(=O)[C@H](CCCCN)NC(=O)[C@@H](CC(C)C)NC1=O]

¹H NMR: (500 MHz, DMSO-d₆) δ8.73 (d, J=8.6 Hz, 2H), 8.01 (d, J=7.4 Hz, 1H), 7.61 (d, J=9.3 Hz, 1H), 7.47-7.39 (m, 2H), 7.34 (t, J=7.6 Hz, 2H), 7.25 (t, J=7.3 Hz, 1H), 7.05 (d, J=7.7 Hz, 1H), 6.27 (s, 1H), 4.46 (q, J=6.7 Hz, 1H), 4.23-4.09 (m, 2H), 4.01 (td, J=7.6, 3.5 Hz, 1H), 3.92 (ddd, J=11.1, 7.8, 3.4 Hz, 1H), 3.71 (d, J=13.6 Hz, 1H), 3.35-3.23 (m, 2H), 3.08 (t, J=8.0 Hz, 1H), 2.81-2.60 (m, 3H), 2.02-1.89 (m, 2H), 1.73-1.40 (m, 12H), 1.39-1.28 (m, 5H), 1.21 (s, 9H), 0.93 (d, J=6.4 Hz, 3H), 0.91-0.82 (m, 6H).

¹³C NMR: (126 MHz, DMSO-d₆) δ174.1, 173.0, 172.7, 172.3, 169.5, 169.4, 135.9, 130.0, 129.0, 126.4, 53.5, 53.2, 50.6, 50.3, 50.0, 48.9, 46.3, 42.2, 39.4, 38.5, 36.4, 30.4, 29.2, 28.2, 27.0, 24.3, 24.1, 23.6, 22.7, 22.7, 22.4, 22.0, 17.5.

HRMS: [M+H]⁺ calculated for C₄₀H₆₇N₈O₆S: 787.4926; found: 787.4942.

UPLC-MS: t_{R} 1.16 min; 99% (UV, PDA, 210-400 nm); (MH⁺) 788.

32

(3S,6S,9R,12S,15R,16S,20aS)-12-(4-aminobutyl)-6-benzyl-N-(tert-butyl)-9-isobutyl-3-methyl-1,4,7,10,13-pentaoxo-15-((phenylthio)methyl)icosahydropyrrolo[1,2-a][1,4,7,10,13,16]hexaazacyclooctadecine-16-carboxamide

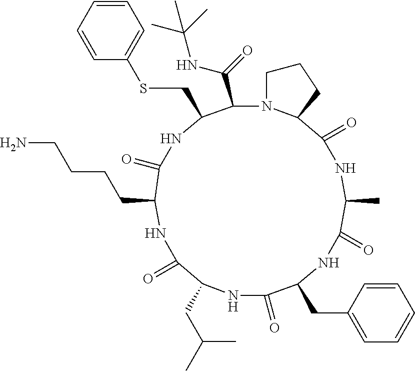 [SMILES: CC(C)C[C@H]1NC(=O)[C@H](Cc2ccccc2)NC(=O)[C@H](C)NC(=O)[C@@H]2CCCN2[C@H](C(=O)NC(C)(C)C)[C@H](CSc2ccccc2)NC(=O)[C@H](CCCCN)NC1=O]

¹H NMR: (500 MHz, DMSO-d₆) δ8.66 (d, J=7.9 Hz, 1H), 8.55 (d, J=3.8 Hz, 1H), 8.01 (d, J=6.7 Hz, 1H), 7.48 (d, J=9.4 Hz, 1H), 7.44-7.39 (m, 2H), 7.37-7.32 (m, 2H), 7.29-7.23 (m, 3H), 7.22-7.17 (m, 1H), 7.17-7.07 (m, 3H), 6.26 (s, 1H), 4.58 (q, J=6.8 Hz, 1H), 4.19-4.05 (m, 2H), 3.97-3.85 (m, 2H), 3.74 (dd, J=14.0, 2.3 Hz, 1H), 3.24 (q, J=7.6 Hz, 1H), 3.17 (dd, J=14.0, 11.3 Hz, 1H), 3.05 (t, J=7.8 Hz, 1H), 3.00 (d, J=6.8 Hz, 2H), 2.82-2.59 (m, 4H), 2.00-1.88 (m, 2H), 1.73-1.28 (m, 11H), 1.25 (d, J=7.4 Hz, 3H), 1.20 (s, 9H), 0.88 (d, J=6.1 Hz, 3H), 0.78 (d, J=6.1 Hz, 3H).

¹³C NMR: (126 MHz, DMSO-d₆) δ174.3, 172.9, 172.7, 171.4, 169.5, 165.9, 136.5, 135.9, 129.9, 129.3, 129.0, 128.0, 126.5, 126.4, 66.8, 66.2, 53.4, 53.1, 53.0, 50.6, 50.2, 49.1, 46.6, 39.4, 38.5, 38.3, 36.2, 30.4, 29.3, 28.1, 27.0, 23.9, 23.6, 22.7, 22.6, 22.0, 17.4.

HRMS: [M+H]⁺ calculated for C₄₃H₆₅N₈O₆S: 821.4770; found: 821.4781.

UPLC-MS: t_{R} 1.20 min; 96% (UV, PDA, 210-400 nm); (MH⁺) 822.

33

(3S,6R,9R,12S,15R,16S,20aS)-12-(4-aminobutyl)-N-(tert-butyl)-3-(4-hydroxybenzyl)-9-isobutyl-6-methyl-1,4,7,10,13-pentaoxo-15-((phenylthio)methyl)icosahydropyrrolo[1,2-a][1,4,7,10,13,16]hexaazacyclooctadecine-16-carboxamide

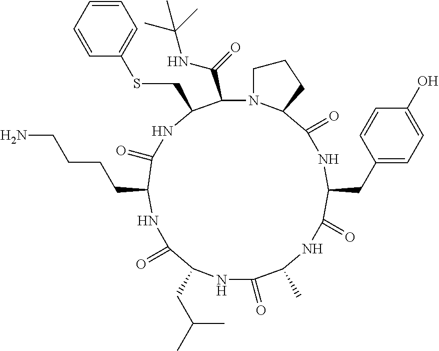 [SMILES: CC(C)C[C@H]1NC(=O)[C@@H](C)NC(=O)[C@H](Cc2ccc(O)cc2)NC(=O)[C@@H]2CCCN2[C@H](C(=O)NC(C)(C)C)[C@H](CSc2ccccc2)NC(=O)[C@H](CCCCN)NC1=O]

¹H NMR: (500 MHz, DMSO-d₆) δ8.75 (d, J=4.8 Hz, 1H), 8.03 (d, J=8.9 Hz, 1H), 7.72 (d, J=6.6 Hz, 1H), 7.50-7.40 (m, 3H), 7.34 (dd, J=8.4, 7.1 Hz, 2H), 7.27-7.19 (m, 1H), 7.12 (d, J=9.2 Hz, 1H), 6.95-6.86 (m, 2H), 6.69-6.60 (m, 2H), 6.49 (s, 1H), 4.50-4.39 (m, 1H), 4.29 (p, J=7.0 Hz, 1H), 4.21 (qd, J=9.1, 3.0 Hz, 1H), 4.14 (ddd, J=10.7, 8.9, 4.0 Hz, 1H), 3.91-3.84 (m, 1H), 3.47 (dd, J=13.6, 3.0 Hz, 1H), 3.21 (d, J=9.0 Hz, 1H), 3.14-3.05 (m, 1H), 3.02 (dd, J=8.9, 6.6 Hz, 1H), 3.00-2.90 (m, 2H), 2.70 (tdd, J=14.9, 7.3, 4.9 Hz, 4H), 1.94-1.81 (m, 2H), 1.73-1.39 (m, 8H), 1.38-1.23 (m, 6H), 1.20 (s, 9H), 0.90 (d, J=6.1 Hz, 3H), 0.86 (d, J=5.9 Hz, 3H).

¹³C NMR: (126 MHz, DMSO-d₆) δ173.5, 172.6, 170.9, 170.5, 169.0, 166.3, 156.1, 136.1, 129.9, 129.4, 129.0, 127.1, 126.2, 115.0, 65.5, 64.8, 53.2, 52.8, 52.5, 50.5, 48.7, 48.1, 46.8, 38.6, 38.5, 36.5, 34.7, 30.3, 29.6, 28.0, 27.0, 24.2, 23.2, 22.8, 22.4, 21.9, 18.5.

HRMS: [M+H]⁺ calculated for C₄₃H₆₅N₈O₇S: 837.4719; found: 837.4724.

UPLC-MS: t_{R} 1.14 min; 99% (UV, PDA, 210-400 nm); (MH⁺) 838.

34

(3S,6R,9S,12S,15R,16S,20aS)-N-(tert-butyl)-3-(4-hydroxybenzyl)-12-(hydroxymethyl)-9-isopropyl-6-methyl-1,4,7,10,13-pentaoxo-15-((phenylthio)methyl)icosahydropyrrolo[1,2-a][1,4,7,10,13,16]hexaazacyclooctadecine-16-carboxamide

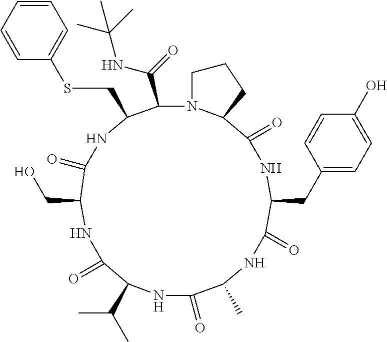 [SMILES: CC(C)[C@@H]1NC(=O)[C@@H](C)NC(=O)[C@H](Cc2ccc(O)cc2)NC(=O)[C@@H]2CCCN2[C@H](C(=O)NC(C)(C)C)[C@H](CSc2ccccc2)NC(=O)[C@H](CO)NC1=O]

¹H NMR: (500 MHz, DMSO-d₆) 6 8.70 (d, J=5.4 Hz, 1H), 8.36 (d, J=7.8 Hz, 1H), 7.47 (m, 2H), 7.39 (dd, J=8.3, 1.4 Hz, 2H), 7.34 (dd, J=8.5, 7.0 Hz, 2H), 7.27-7.16 (m, 1H), 6.99 (s, 1H), 6.94-6.85 (m, 2H), 6.62 (d, J=8.5 Hz, 2H), 6.57 (d, J=9.4 Hz, 1H), 4.87 (s, 1H), 4.64-4.53 (m, 2H), 4.27 (tt, J=10.5, 5.7 Hz, 1H), 3.99 (s, 1H), 3.73 (td, J=14.3, 12.7, 7.2 Hz, 2H), 3.65 (dd, J=7.0, 5.4 Hz, 1H), 3.21-3.07 (m, 3H), 3.05-2.94 (m, 2H), 2.89 (dd, J=12.9, 6.7 Hz, 1H), 2.76 (ddd, J=9.6, 6.5, 3.6 Hz, 1H), 2.50 (m, 1H), 2.01 (m, 1H), 1.84 (tt, J=12.4, 4.7 Hz, 1H), 1.63-1.43 (m, 2H), 1.31 (d, J=7.0 Hz, 3H), 1.22 (m, 10H), 0.97 (dd, J=17.9, 6.8 Hz, 6H).

¹³C NMR: (126 MHz, DMSO-d₆) δ174.2, 172.7, 171.2, 169.7, 168.9, 168.0, 155.9, 136.4, 130.0, 129.1, 129.0, 127.5, 126.2, 114.9, 64.2, 64.1, 61.8, 60.6, 56.6, 52.2, 50.7, 48.0, 47.8, 46.2, 36.6, 35.1, 29.2, 29.0, 28.1, 23.4, 19.8, 19.1, 19.0.

HRMS: [M+H]⁺ calculated for C₃₉H₅₆N₇O₈S: 782.3906; found: 782.3916.

LC-MS: t_{R} 1.44 min; 98% (UV, PDA, 210-400 nm); (MH⁺) 782.

sub2-949 AT-04-55 32 now 35

(3S,6R,9S,12S,15S,16R,20aR)-9-((1H-indol-3-yl)methyl)-12-(3-amino-3-oxopropyl)-3-((S)-sec-butyl)-N-(tert-butyl)-6-methyl-1,4,7,10,13-pentaoxo-15-((phenylthio)methyl)icosahydropyrrolo[1,2-a][1,4,7,10,13,16]hexaazacyclooctadecine-16-carboxamide

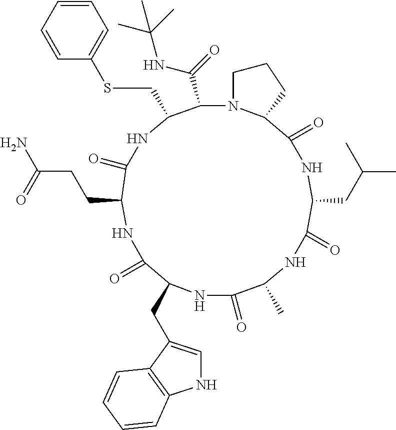 [SMILES: CC(C)C[C@H]1NC(=O)[C@H]2CCCN2[C@@H](C(=O)NC(C)(C)C)[C@@H](CSc2ccccc2)NC(=O)[C@H](CCC(N)=O)NC(=O)[C@H](Cc2c[nH]c3ccccc23)NC(=O)[C@@H](C)NC1=O]

¹H NMR: (500 MHz, DMSO-d₆) δ10.80 (d, J=2.4 Hz, 1H), 9.38 (d, J=9.3 Hz, 1H), 9.08 (s, 1H), 8.41 (d, J=7.4 Hz, 1H), 8.26 (d, J=8.6 Hz, 1H), 8.09 (d, J=6.9 Hz, 1H), 7.72 (s, 1H), 7.64 (dq, J=7.9, 0.7 Hz, 1H), 7.44-7.22 (m, 7H), 7.12 (d, J=2.4 Hz, 1H), 7.04 (ddd, J=8.2, 7.0, 1.2 Hz, 1H), 6.96 (ddd, J=8.0, 7.0, 1.0 Hz, 1H), 6.85-6.79 (m, 1H), 4.63 (ddd, J=10.2, 8.5, 4.0 Hz, 1H), 4.35-4.26 (m, 2H), 4.17 (ddd, J=8.6, 7.4, 5.4 Hz, 1H), 4.09 (t, J=8.4 Hz, 1H), 3.93 (td, J=7.0, 4.1 Hz, 1H), 3.79 (d, J=4.1 Hz, 1H), 3.37 (dd, J=13.8, 6.1 Hz, 1H), 3.24-3.09 (m, 2H), 2.94 (t, J=7.4 Hz, 1H), 2.87 (dd, J=14.7, 10.2 Hz, 1H), 2.72-2.62 (m, 1H), 2.41-2.31 (m, 1H), 2.17 (ddd, J=8.8, 6.7, 2.4 Hz, 2H), 2.02-1.80 (m, 3H), 1.78-1.52 (m, 3H), 1.50-1.40 (m, 1H), 1.22 (s, 9H), 1.17-1.07 (m, 1H), 0.93 (d, J=6.9 Hz, 3H), 0.83-0.75 (m, 6H).

¹³C NMR: (126 MHz, DMSO-d₆) δ173.5, 173.2, 171.7, 171.1, 167.9, 167.1, 165.9, 136.1, 134.9, 129.4, 128.7, 127.3, 126.6, 123.8, 120.8, 118.6, 118.1, 111.2, 109.9, 58.6, 58.1, 55.5, 52.8, 52.0, 51.9, 50.5, 48.4, 48.0, 35.9, 33.5, 31.6, 31.4, 28.2, 28.0, 26.8, 23.8, 23.4, 18.4, 14.7, 10.2.

HRMS: [M+H]⁺ calculated for C₄₄H₆₂N₉O₇S: 860.4487; found: 860.4479.

LC-MS: t_{R} 1.42 min; 95% (UV, PDA, 210-400 nm); (MNa⁺) 861.

TABLE 1
Macrocycles synthesized on solid phase by three-component coupling

| | | | | Isolated | | |
|---|---|---|---|---|---|---|
| | | Ring | Mw | mass | Yield | Purity |
| Compound | Structure | size | (g·mol⁻¹)^{a} | (mg)^{b} | (%)^{c} | (%)^{d} |
| 14 | 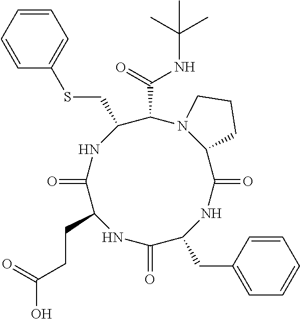 [SMILES: CC(C)(C)NC(=O)[C@H]1[C@@H](CSc2ccccc2)NC(=O)[C@H](CCC(=O)O)NC(=O)[C@@H](Cc2ccccc2)NC(=O)[C@H]2CCCN21] | 12 | 637.8 | 10 | 16 | 96 |
| 15 | 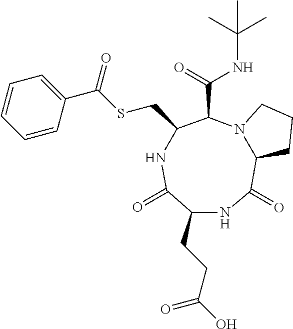 [SMILES: CC(C)(C)NC(=O)[C@@H]1[C@H](CSC(=O)c2ccccc2)NC(=O)[C@H](CCC(=O)O)NC(=O)[C@@H]2CCCN12] | 9 | 518.6 | 7 | 13 | 98 |
| 16 | 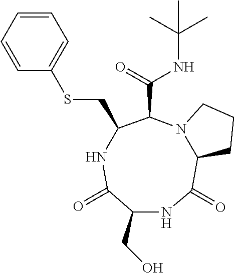 [SMILES: CC(C)(C)NC(=O)[C@@H]1[C@H](CSc2ccccc2)NC(=O)[C@H](CO)NC(=O)[C@@H]2CCCN12] | 9 | 448.6 | 8 | 13 | 94 |
| 17 | 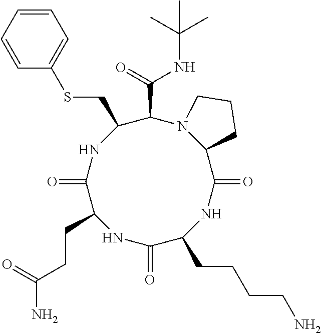 [SMILES: CC(C)(C)NC(=O)[C@@H]1[C@H](CSc2ccccc2)NC(=O)[C@H](CCC(N)=O)NC(=O)[C@H](CCCCN)NC(=O)[C@@H]2CCCN12] | 12 | 617.8 | 13 | 15 | 95 |
| 18 | 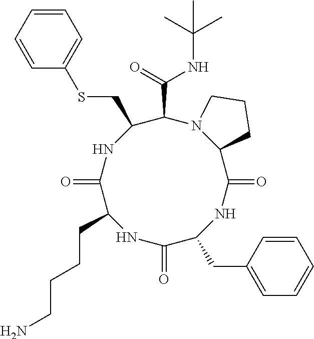 [SMILES: CC(C)(C)NC(=O)[C@@H]1[C@H](CSc2ccccc2)NC(=O)[C@H](CCCCN)NC(=O)[C@@H](Cc2ccccc2)NC(=O)[C@@H]2CCCN12] | 12 | 636.9 | 7 | 8 | 90 |
| 19 | 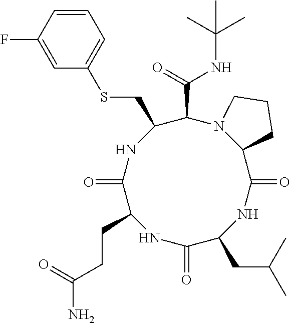 [SMILES: CC(C)C[C@@H]1NC(=O)[C@@H]2CCCN2[C@H](C(=O)NC(C)(C)C)[C@H](CSc2cccc(F)c2)NC(=O)[C@H](CCC(N)=O)NC1=O] | 12 | 620.8 | 5 | 7 | 93 |
| 20 | 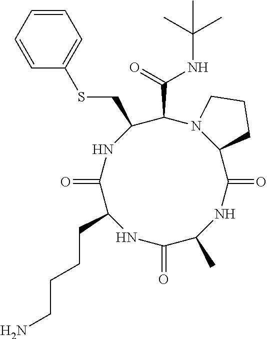 [SMILES: C[C@@H]1NC(=O)[C@@H]2CCCN2[C@H](C(=O)NC(C)(C)C)[C@H](CSc2ccccc2)NC(=O)[C@H](CCCCN)NC1=O] | 12 | 560.8 | 16 | 20 | 97 |
| 21 | 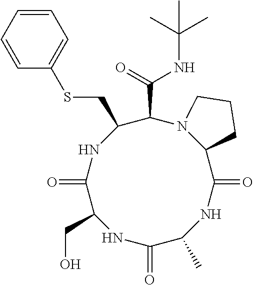 [SMILES: C[C@H]1NC(=O)[C@@H]2CCCN2[C@H](C(=O)NC(C)(C)C)[C@H](CSc2ccccc2)NC(=O)[C@H](CO)NC1=O] | 12 | 519.7 | 10 | 16 | 96 |
| 22 | 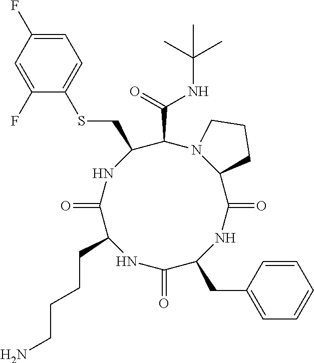 [SMILES: CC(C)(C)NC(=O)[C@@H]1[C@H](CSc2ccc(F)cc2F)NC(=O)[C@H](CCCCN)NC(=O)[C@H](Cc2ccccc2)NC(=O)[C@@H]2CCCN12] | 12 | 672.8 | 7 | 7 | 95 |
| 23 | 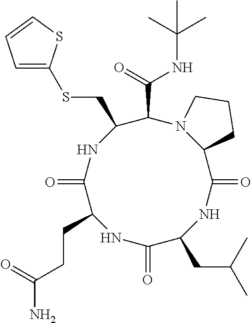 [SMILES: CC(C)C[C@@H]1NC(=O)[C@@H]2CCCN2[C@H](C(=O)NC(C)(C)C)[C@H](CSc2cccs2)NC(=O)[C@H](CCC(N)=O)NC1=O] | 12 | 608.8 | 7 | 10 | 90 |
| 24 | 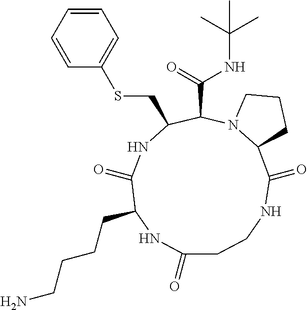 [SMILES: CC(C)(C)NC(=O)[C@@H]1[C@H](CSc2ccccc2)NC(=O)[C@H](CCCCN)NC(=O)CCNC(=O)[C@@H]2CCCN12] | 13 | 560.8 | 25 | 31 | 99 |
| 25 | 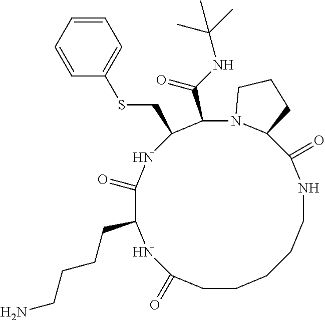 [SMILES: CC(C)(C)NC(=O)[C@@H]1[C@H](CSc2ccccc2)NC(=O)[C@H](CCCCN)NC(=O)CCCCCNC(=O)[C@@H]2CCCN12] | 16 | 602.8 | 10 | 12 | 98 |
| 26 | 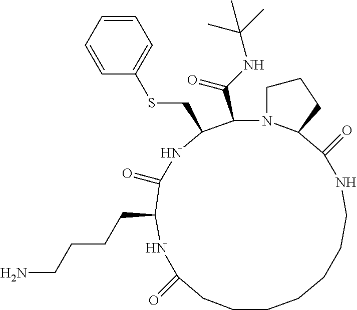 [SMILES: CC(C)(C)NC(=O)[C@@H]1[C@H](CSc2ccccc2)NC(=O)[C@H](CCCCN)NC(=O)CCCCCCCNC(=O)[C@@H]2CCCN12] | 18 | 630.9 | 14 | 15 | 91 |
| 27 | 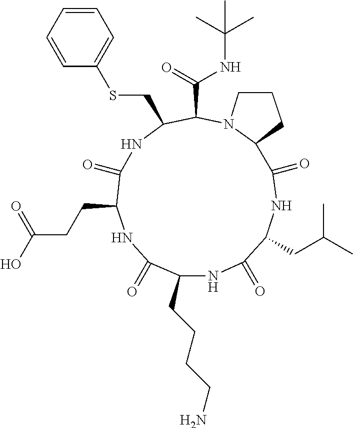 [SMILES: CC(C)C[C@H]1NC(=O)[C@@H]2CCCN2[C@H](C(=O)NC(C)(C)C)[C@H](CSc2ccccc2)NC(=O)[C@H](CCC(=O)O)NC(=O)[C@H](CCCCN)NC1=O] | 15 | 732.0 | 7 | 19 | 91 |
| 28 | 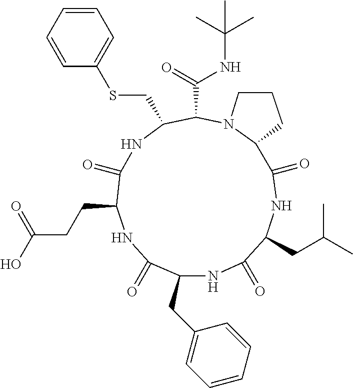 [SMILES: CC(C)C[C@@H]1NC(=O)[C@H]2CCCN2[C@@H](C(=O)NC(C)(C)C)[C@@H](CSc2ccccc2)NC(=O)[C@H](CCC(=O)O)NC(=O)[C@H](Cc2ccccc2)NC1=O] | 15 | 751.0 | 9 | 12 | 95 |
| 29 | 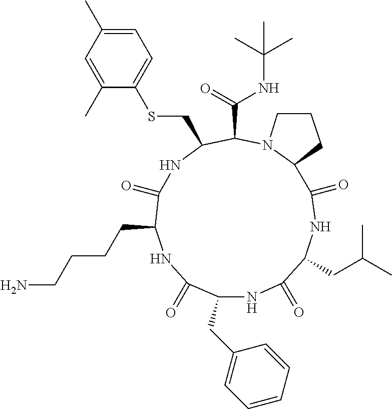 [SMILES: Cc1ccc(SC[C@@H]2NC(=O)[C@H](CCCCN)NC(=O)[C@@H](Cc3ccccc3)NC(=O)[C@@H](CC(C)C)NC(=O)[C@@H]3CCCN3[C@@H]2C(=O)NC(C)(C)C)c(C)c1] | 15 | 778.1 | 9 | 9 | 94 |
| 30 | 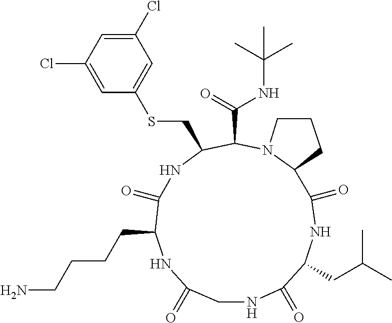 [SMILES: CC(C)C[C@H]1NC(=O)[C@@H]2CCCN2[C@H](C(=O)NC(C)(C)C)[C@H](CSc2cc(Cl)cc(Cl)c2)NC(=O)[C@H](CCCCN)NC(=O)CNC1=O] | 15 | 728.8 | 7 | 7 | 95 |
| 31 | 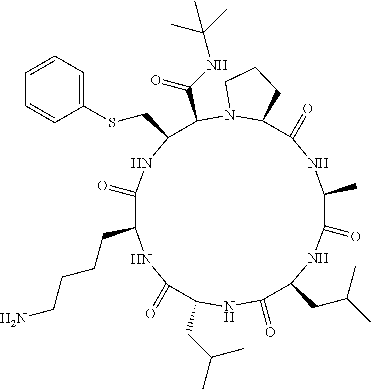 [SMILES: CC(C)C[C@@H]1NC(=O)[C@H](C)NC(=O)[C@@H]2CCCN2[C@H](C(=O)NC(C)(C)C)[C@H](CSc2ccccc2)NC(=O)[C@H](CCCCN)NC(=O)[C@@H](CC(C)C)NC1=O] | 18 | 787.1 | 7 | 8 | 99 |
| 32 | 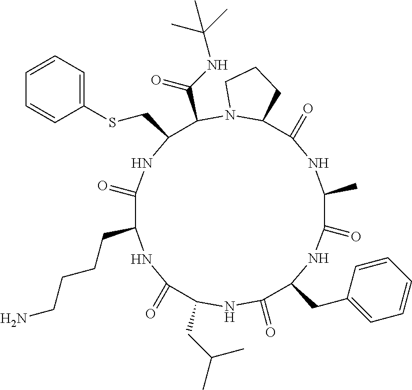 [SMILES: CC(C)C[C@H]1NC(=O)[C@H](Cc2ccccc2)NC(=O)[C@H](C)NC(=O)[C@@H]2CCCN2[C@H](C(=O)NC(C)(C)C)[C@H](CSc2ccccc2)NC(=O)[C@H](CCCCN)NC1=O] | 18 | 821.1 | 17 | 18 | 96 |
| 33 | 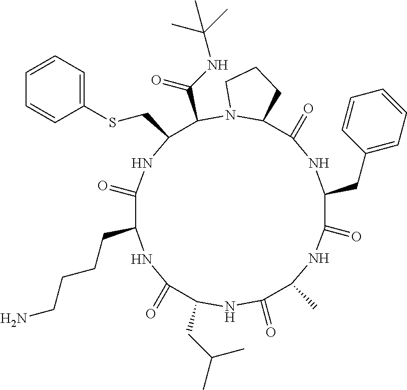 [SMILES: CC(C)C[C@H]1NC(=O)[C@@H](C)NC(=O)[C@H](Cc2ccccc2)NC(=O)[C@@H]2CCCN2[C@H](C(=O)NC(C)(C)C)[C@H](CSc2ccccc2)NC(=O)[C@H](CCCCN)NC1=O] | 18 | 837.1 | 16 | 18 | 99 |
| 34 | 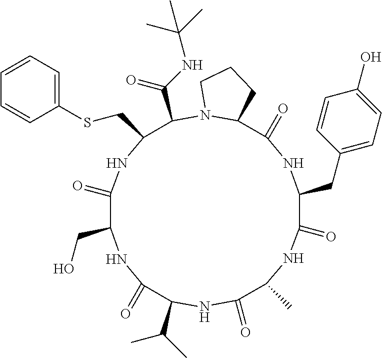 [SMILES: CC(C)[C@@H]1NC(=O)[C@@H](C)NC(=O)[C@H](Cc2ccc(O)cc2)NC(=O)[C@@H]2CCCN2[C@H](C(=O)NC(C)(C)C)[C@H](CSc2ccccc2)NC(=O)[C@H](CO)NC1=O] | 18 | 782.0 | 8 | 10 | 98 |
| 35 | 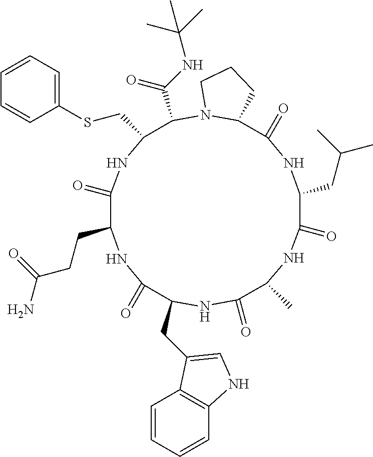 [SMILES: CC(C)C[C@H]1NC(=O)[C@H]2CCCN2[C@@H](C(=O)NC(C)(C)C)[C@@H](CSc2ccccc2)NC(=O)[C@H](CCC(N)=O)NC(=O)[C@H](Cc2c[nH]c3ccccc23)NC(=O)[C@@H](C)NC1=O] | 18 | 860.1 | 7 | 7 | 95 |

^{a}MW of free base;
^{b}Isolated as a TFA (14-30) or formate (31-35) salt;
^{c}Yields of 14-30 were calculated considering one TFA equivalent per net positive charge on the macrocycle, and 31-35 using formate as a counter-anion;
^{d}As determined by LC-MS (14-30) or UPLC-MS (31-35).

Although preferred embodiments of the invention have been described herein, it will be understood by those skilled in the art that variations may be made thereto without departing from the spirit of the invention or the scope of the appended claims. A person skilled in the art would further understand that the present disclosure is not limited to the specific combinations described herein as embodiments, and that embodiments, and each characteristic or feature thereof, may be combined in any suitable and/or reasonable manner.

All references mentioned herein, including in the following reference list, are incorporated by reference in their entirety.

### REFERENCES

1. Driggers, E. M., et al., The exploration of macrocycles for drug discovery—an underexploited structural class. Nat Rev Drug Discov, 2008. 7(7): p. 608-24.
2. Oyelere, A. K., Macrocycles in medicinal chemistry and drug discovery. Curr Top Med Chem, 2010. 10(14): p. 1359-60.
3. Marsault, E. and M. L. Peterson, Macrocycles are great cycles: applications, opportunities, and challenges of synthetic macrocycles in drug discovery. J Med Chem, 2011. 54(7): p. 1961-2004.
4. White, C. J. and A. K. Yudin, Contemporary strategies for peptide macrocyclization. Nat Chem, 2011. 3(7): p. 509-24.
5. Giordanetto, F. and J. Kihlberg, Macrocyclic drugs and clinical candidates: what can medicinal chemists learn from their properties? J Med Chem, 2014. 57(2): p. 278-95.
6. Yudin, A., commentary.
7 Yudin, A. K., Macrocycles: lessons from the distant past, recent developments, and future directions. Chem. Sci., 2015.
8. Villar, E. A., et al., How proteins bind macrocycles. Nat Chem Biol, 2014. 10(9): p. 723-31.
9. Falchi, F., F. Caporuscio, and M. Recanatini, Structure-based design of small-molecule protein-protein interaction modulators: the story so far. Future Med Chem, 2014. 6(3): p. 343-57.
10. Marsault, E., et al., Efficient parallel synthesis of macrocyclic peptidomimetics. Bioorg Med Chem Lett, 2008. 18(16): p. 4731-5.
11. Tse, B. N., et al., Translation of DNA into a library of 13,000 synthetic small-molecule macrocycles suitable for in vitro selection. J Am Chem Soc, 2008. 130(46): p. 15611-26.
12. Jefferson, E. A., et al., New inhibitors of bacterial protein synthesis from a combinatorial library of macrocycles. J Med Chem, 2002. 45(16): p. 3430-9.
13. Bogdan, A. R., et al., Strained cyclophane macrocycles: impact of progressive ring size reduction on synthesis and structure. J Am Chem Soc, 2012. 134(4): p. 2127-38.
14. Enck, S. K., F.; Marahiel, M. A.; Geyer, A., ChemBioChem, 2008. 9: p. 2597-2601.
15. Delorbe, J. E., et al., Thermodynamic and Structural Effects of Macrocyclization as a Constraining Method in Protein-Ligand Interactions. ACS Med Chem Lett, 2010. 1(8): p. 448-452.
16. Hili, R., V. Rai, and A. K. Yudin, Macrocyclization of linear peptides enabled by amphoteric molecules. J Am Chem Soc, 2010. 132(9): p. 2889-91.
17. Li, X. and A. K. Yudin, Epimerization-and protecting-group-free synthesis of peptidomimetic conjugates from amphoteric amino aldehydes. J Am Chem Soc, 2007. 129(46): p. 14152-3.
18. Yudin, A. K. and R. Hili, Overcoming the demons of protecting groups with amphoteric molecules. Chemistry, 2007. 13(23): p. 6538-42.
19. Scully, C. C. G. R., V.; Poda, G.; Zaretsky, S.; Burns, D. C.; Houliston, R. S.; Lou, T.; Yudin, A. K. , Chem.—A Eur. J. , 2012. 18: p. 15612.
20. Zaretsky, S., et al., Exocyclic control of turn induction in macrocyclic peptide scaffolds. Chemistry: Eur J, 2013. 19(52): p. 17668-72.
21. Chung, B. K. W. H., J. L.; Scully, C. C. G.; Zaretsky, S.; Yudin, A. K., MedChemComm, 2013.4: p. 1124.
22. Zaretsky, S. T., J.; Hickey, J. L.; Yudin, A. K. , Methods in Molecular Biology (Springer): Peptide Libraries.; Derda, R., Ed.; , 2014. 6: p. in press.
23. Kates, S. A., F, Solid-phase synthesis: a practical guide; CRC Press. 2000.
24. Bunin, B. A. E., J A., J. Am. Chem. Soc., 1992. 114: p. 10997.
25. Treder, A. P., et al., Solid-Phase Synthesis of Piperazinones via Disrupted Ugi Condensation. Org Lett, 2014.
26. Dagmar, C. K., D. C.; Bräse, S., Synlett, 2011: p. 161-164.
27. Nishiguchi, T. F., S.; Kuroda, M.; Kajisaki, K.; Saitoh, M., J. Org. Chem., 1998. 63: p. 8183-8187.
28. Rosse, G. O., F.; Schröder, H., J. Comb. Chem., 1999. 1: p. 397-401.
29. Conroy, T., K. A. Jolliffe, and R. J. Payne, Efficient use of the Dmab protecting group: applications for the solid-phase synthesis of N-linked glycopeptides. Org Biomol Chem, 2009. 7(11): p. 2255-8.
30. Rijkers, D. T., J. A. den Hartog, and R. M. Liskamp, Synthesis and biological activity of N-terminal lipidated and/or fluorescently labeled conjugates of astressin as corticotropin releasing factor antagonists. Bioorg Med Chem, 2004. 12(19): p. 5099-106.
31. Kates, S. A. A., F., Solid Phase Synthesis, a Practical Guide; Marcel Dekker Inc. 2000.
32. Meutermans, W. D., et al., Difficult macrocyclizations: new strategies for synthesizing highly strained cyclic tetrapeptides. Org Lett, 2003. 5(15): p. 2711-4.
33. Zaretsky, S. A., S.; Rotstein, B. H.; Hickey, J. L.; Conor, C. G.; Denis, J. D. S.; Courtemanche, R.; Yu, J. C. Y.; Chung, B. K. W.; Yudin, A. K. , submitted.

34. http://www.silicycle.com/ca/products/silicycle-miniblock-xt.

1. A process to produce a cyclic molecule bound to a solid support comprising reacting a peptide, having an amino terminus and a carboxyl terminus and bound to the solid support by a side chain of the peptide, with an isocyanide and a compound having formula (Ia) and/or (Ib): 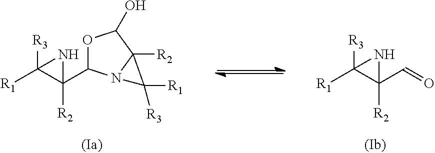
wherein:
R₁, R₂ and R₃ are independently selected from H; lower alkyl; alkenyl; heterocycle; cyckoalkyl; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -lower alkyl-aryl, or —NRₐR_{b}, where Rₐ and R_{b} are independently selected from H, lower alkyl, aryl or -lower alkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents; and
the aldehyde component thereof may optionally be in its bisulfite adduct form;
and the compound comprises an aziridine chiral center proximal to the aldehyde with stereochemistry that is homochiral with respect to the carbon atom proximal to the amino terminus of the peptide.

2. The process of claim 1, wherein the amino terminus of the peptide is a secondary amino group.

3. The process of claim 1, wherein any one of R₁-R₃ is H.

4. The process of claim 1, wherein R₁-R₃ is H.

5. The process of claim 1, wherein R₂ and R₃ are H.

6. The process of claim 5, wherein R₁ is CH₂OTBDMS or CH₂ⁱPr.

7. The process of claim 1, wherein the isocyanide is selected from the group consisting of: (S)-(−)-α-Methylbenzyl isocyanide; 1,1,3,3,-Tetramethylbutyl isocyanide; 1-Pentyl isocyanide; 2,6-Dimethylphenyl isocyanide; 2-Morpholinoethyl isocyanide; 2-Naphthyl isocyanide; 2-Pentyl isocyanide; 4-Methoxyphenyl isocyanide; Benzyl isocyanide; Cutyl isocyanide; Cyclohexyl isocyanide; Isopropyl isocyanide; p-Toluenesulfonylmethyl isocyanide; Phenyl isocyanide dichloride; tert-Butyl isocyanide; (Trimethylsilyl)methyl isocyanide; 1H-Benzotriazol-1-ylmethyl isocyanide; 2-Chloro-6-methylphenyl isocyanide; Di-tert-butyl 2-isocyanosuccinate; tert-Butyl 2-isocyano-3-methylbutyrate; tert-Butyl 2-isocyano-3-phenylpropionate; tert-Butyl 2-isocyanopropionate; and tert-Butyl 3-isocyanopropionate; tert-Butyl isocyanoacetate; and ethyl isocyanoacetate.

8. The process of claim 1, wherein the isocyanide is tert-Butyl isocyanide.

9. The process of claim 1, wherein the peptide is between 2 and 30 amino acids in length.

10. The process of claim 1, wherein the peptide is between 2 and 9 amino acids in length.

11. The process of claim 1, wherein the cyclic molecule bound to the solid support is of formula [(II)]: 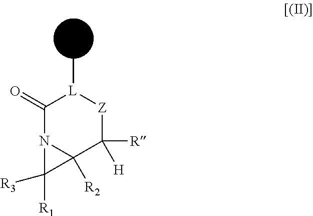
wherein,
Z is an amino terminus of the peptide;
—C═O— is the carboxy terminus of the peptide; and
L, along with Z and —C═O— is the peptide;
R″ is an optionally substituted amide.

12. A process for preparing a cyclic molecule of formula [(III)] bound to a solid support: 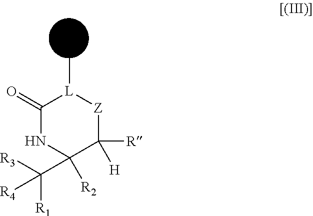
wherein,
R₁, R₂ and R₃ are independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or —NRₐR_{b}, where Rₐ and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents,
Z is an amino terminus of a peptide;
—C=O— is the carboxy terminus of the peptide;
L, along with Z and —C=O— is the peptide;
R″ is an optionally substituted amide;
R₄ is a nucleophile;
wherein the peptide is bound to the solid support by a side chain of the peptide;
comprising reacting a compound having formula [II] bound to the solid support with a nucleophile:

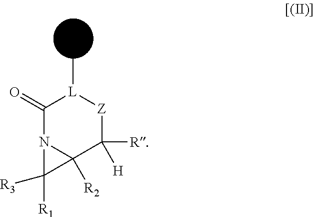

13. A process for preparing a cyclic molecule of formula [(IV)]: 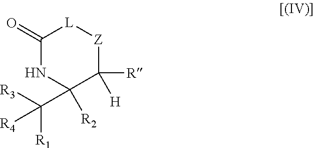
wherein,
R₁, R₂ and R₃ are independently selected from H; lower alkyl; aryl; heteroaryl; alkenyl; heterocycle; esters of the formula —C(O)OR^{*} wherein R^{*} is selected from alkyl and aryl; amides of the formula —C(O)NR^{**}R^{***}, wherein R^{**} and R^{***} are independently selected from alkyl and aryl; —CH₂C(O)R, wherein R is selected from —OH, lower alkyl, aryl, -loweralkyl-aryl, or NRₐR_{b}, where R and R_{b} are independently selected from H, lower alkyl, aryl or -loweralkyl-aryl; —C(O)R_{c}, wherein R_{c} is selected from lower alkyl, aryl or -lower alkyl-aryl; or -lower alkyl-OR_{d}, wherein R_{d} is a suitable protecting group or OH group; all of which are optionally substituted at one or more substitutable positions with one or more suitable substituents;
Z is an amino terminus of a peptide;
—C=O— is the carboxy terminus of the peptide;
L, along with Z and —C=O— is the peptide;
R″ is an optionally substituted amide;
R₄ is a nucleophile;
comprising cleaving a solid support from a cyclic molecule of formula [(III)] bound to the solid support: 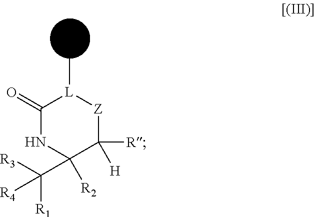
and optionally deprotecting one or more side chains of the peptide if one or more of said side chains are protected with protecting groups.

14. The process of claim 1, wherein the solid support is a resin.

15. The process of claim 14, wherein the resin is selected from the group consisting of Wang, MBHA, HMPA, Tentagel, Trityl, 2′-Chlorotrityl, Argogel, PS-PEG, ChemMatrix, PEG support, Mimotopes' Lanterns.

16. The process of claim 1, wherein the peptide is elongated prior to cyclization.

17. The process of claim 16, wherein elongation is by Fmoc chemistry .

18. The process of claim 1, further comprising ring-opening of the aziridine moiety with a nucleophile.

19. The process of claim 1, further comprising conjugating a fluorescent tag to the cyclic molecule by nucleophilic ring-opening of the aziridine moiety.

20. The process of claim 12, wherein the nucleophile is selected from the group consisting of: R—C(O)SH, Ar—C(O)SH, Ar—SH, H₂, R—SH, RS—, N₃—, R₃P, NC—, I—, Ar—NH2, Br—, R—CO2H; preferentially, the nucleophile is selected from the group consisting of: R—C(O)SH, Ar—C(O)SH, Ar—SH, H₂, N₃

21. The process of claim 12, wherein the nucleophilic ring-opening of the aziridine moiety is carried out using a soft, highly polarizable, low-electronegativity nucleophile having pKa<15.

22. The process of claim 1, further comprising cleavage of the solid support from the cyclic molecule.

23. The process of claim 1, further comprising deprotecting one or more side chains of the amino acid molecule if one or more of said side chains are protected with protecting groups.

24. The process of claim 1, wherein the peptide is a linear peptide.

25. The process of claim 24, wherein the amino terminus amino acid of the linear peptide is selected from the group consisting of proline and an amino acid with an amino group substituted with H and Bn, with H and CH2CH2SO2Ph, with H and CH2CH2CN, or with H and CH3.

26. The process of claim 1, wherein the amino acids of the peptide are D or L amino acids selected from the group consisting of: alanine, arginine, asparagine, aspartic acid, cysteine, glutamic acid, glutamine, glycine, histidine, isoleucine, leucine, lysine, methionine, phenylalanine, proline, selenocysteine, serine, tyrosine, threonine, tryptophan and valine.

27. The process of claim 1, wherein the amino acids of the peptide are alpha-amino acids.

28. The process of claim 1 wherein the amino acids of the peptide are beta-amino acids.

29. The process of claim 1 wherein the amino acids of the peptide are gamma-amino acids.

30. The process of claim 1, wherein the solid support is bound to the side chain of the carboxy terminus of the peptide.

31. The process of claim 1, wherein the side chains of the peptide are polar side chains.

32. The process of claim 1, wherein the side chains of the peptide are non-polar side chains.

33. The process of claim 1, wherein the side chain of the amino acid molecule is selected from the group consisting of: glutamic acid, glutamine, serine, threonine, lysine, aspartic acid, asparagine, homo-serine, ornithine, 2,4-diaminobutyric acid, 2,3-diaminopropionic acid, tyrosine, tryptophan, histidine, and 4-hydroxy-proline.

34.-35. (canceled)
